(19) United States
(12) Patent Application Publication
(10) Pub. No.: US 2026/0306006 A1
(43) Pub. Date: Oct. 8, 2026

(54) MAPPING AND NAVIGATION SYSTEM

(71) Applicant: ENCHANNEL MEDICAL LTD., Irvine, CA (US)

(72) Inventors: Graydon Ernest Beatty, San Diego, CA (US); Min Zhu, Coto De Caza, CA (US); Jian Ni, Rancho Mission Viejo, CA (US); Morteza Hakimi Siboni, Rancho Santa Margarita, CA (US); Zhongwei Zhi, Foothill Ranch, CA (US); Kevin Justin Manalastas Herrera, Santa Ana, CA (US); Shichen Xu, Irvine, CA (US); J. Christopher Flaherty, Nottingham, NH (US); R. Maxwell Flaherty, Topsfield, MA (US)

(21) Appl. No.: 19/484,911

(22) PCT Filed: May 22, 2024
(86) PCT No.: PCT/US2024/030585

§ 371 Date: Nov. 17, 2025

Related U.S. Application Data
(60) Provisional application No. 63/503,665, filed on May 22, 2023.
(60) Provisional application No. 63/506,314, filed on Jun. 5, 2023.

(51) Int. Cl. A61B 5/367; A61B 5/00; A61B 5/287

(52) U.S. Cl. CPC A61B 5/367; A61B 5/287; A61B 5/6858

(57) ABSTRACT

Provided herein are systems, devices, and methods for performing a medical procedure on a patient. The system includes a catheter device and a console for operating the catheter device. The catheter device includes a functional assembly that is located on the distal portion of the catheter and includes one or more electrodes. The console includes a diagnostic module for recording signals from the electrodes and a processing unit for processing the recorded signals.

### CROSS-REFERENCE TO RELATED APPLICATIONS

This application claims the benefit of: U.S. Provisional Patent Application Ser. No. 63/503,665 (Docket No. ENC-001-PR1), entitled “Basket Catheter Comprised of Flex Circuits with Electrodes on Both Sides of the Splines”, filed May 22, 2023; and U.S. Provisional Patent Application Ser. No. 63/506,314 (Docket No. ENC-001-PR2), entitled “Hybrid Navigation of the Catheter Using Both Magnetic and Impedance Fields”, filed Jun. 5, 2023; the contents of each of which is incorporated herein by reference in its entirety for all purposes.

### FIELD OF THE INVENTIVE CONCEPTS

The present inventive concepts relate generally to systems, devices, and methods for diagnosing cardiac disease, and in particular mapping cardiac electrical activity.

### BACKGROUND

Several medical procedures include treating cardiac disease using catheter devices that are inserted proximate the heart of a patient (e.g., inserted into a heart chamber). Identifying diseased tissue and tracking the location and orientation of various devices used to identify and/or treat the tissue can be difficult, and this difficulty can cause poor clinical results.

There is a need for systems, methods, and devices that improve diagnosis and treatment of cardiac disease.

### SUMMARY

According to an aspect of the present inventive concepts, a system for performing a medical procedure on a patient comprises: a catheter device comprising a distal portion including a distal end and a functional assembly located on the distal portion of the catheter, the functional assembly comprising one or more electrodes. The system further comprises a console for operating the catheter device comprising: a diagnostic module configured to record signals from at least one of the one or more electrodes; and a processing unit configured to process the recorded signals.

In some embodiments, the functional assembly comprises a distal surface, and the distal surface comprises the distal end of the catheter device.

In some embodiments, the catheter device further comprises a magnetic sensor. The magnetic sensor can comprise two or more magnetic sensors. The functional assembly can comprise the magnetic sensor. The magnetic sensor can be configured to provide a signal related to the location, orientation, and/or geometric configuration of the functional assembly. The magnetic sensor can comprise a five degree of freedom magnetic sensor. The magnetic sensor can comprise a six degree of freedom magnetic sensor.

In some embodiments, the functional assembly further comprises multiple splines, and each spline of the multiple splines comprises at least one of the one or more electrodes. The multiple splines can comprise at least four splines. The multiple splines can comprise at least eight splines. Each of the multiple splines can comprise a flat sheet. Each of the multiple splines can comprise a material selected from the group consisting of: nickel-titanium alloy; stainless steel; polyethylenimine (PEI); polyimide; and combinations thereof. Each spline of the multiple splines can comprise a first side and a second side.

In some embodiments, the one or more electrodes comprise a first set of one or more electrodes positioned on the first side of a first spline of the multiple splines and a second set of one or more electrodes positioned on the second side of the first spline.

In some embodiments, each of the first and second sets of one or more electrodes comprise at least three, six, eight, ten, twelve, fourteen, or sixteen electrodes.

In some embodiments, each electrode of the first set of electrodes comprises a corresponding electrode in the second set of electrodes, and the corresponding electrodes comprise a pair.

In some embodiments, each pair of electrodes from the first set of electrodes and second set of electrodes are axially aligned along the length of the first spline.

In some embodiments, each pair of electrodes from the first set of electrodes and second set of electrodes are axially offset along the length of the first spline. The system can further comprise one or more flex circuits, and each flex circuit can be attached to a respective spline of the multiple splines, and each spline can comprise at least one of the one or more electrodes. Each flex circuit can be bonded and/or laminated to the respective spline. Each spline of the multiple splines can comprise a first side and a second side. The one or more flex circuits can comprise a first flex circuit attached to the first side of a first spline of the multiple splines and a second flex circuit attached to the second side of the first spline. The one or more flex circuits can comprise a first flex circuit comprising a first portion and a second portion, and the first portion can be attached to the first side of a first spline of the multiple splines, and the second portion can be attached to the second side of the first spline. The multiple electrodes can comprise a first set of one or more electrodes positioned on the first portion of the flex circuit and a second set of one or more electrodes positioned on the second portion of the flex circuit. The system can further comprise a control assembly comprising a puller tube, and the puller tube can be configured to radially expand and/or contract the functional assembly. The control assembly can further comprise a housing and the functional assembly can comprise a distal end, and the housing can connect the puller tube to the distal end of the functional assembly. The housing can comprise a first navigation element. The first navigation element can comprise a magnetic sensor. The catheter device can further comprise a second navigation element. The second navigation element can comprise a magnetic sensor. The second navigation element can be positioned proximate and proximal to the functional assembly. The catheter device can further comprise a shaft comprising a distal end and a coupler housing positioned proximate the distal end, and the coupler housing can connect the functional assembly to the distal end of the shaft, and the second navigation element can be positioned within the coupler housing.

In some embodiments, the functional assembly comprises a diameter of at least 12 mm, no more than 35 mm, or both.

In some embodiments, the one or more electrodes comprise a material selected from the group consisting of: gold; platinum; platinum-iridium; iridium oxide; PDOT conductive polymer; titanium nitride; graphene; a precious metal alloy; and combinations of these.

In some embodiments, the one or more electrodes comprise a coating selected from the group consisting of: a gold coating; a coating configured to decrease the input impedance of the electrode; a PDOT coating; an iridium oxide coating; a titanium nitride coating; an oxide coating; and combinations thereof.

In some embodiments, the catheter device comprises at least a first catheter, and the system further comprises: a navigation sub-system comprising: a magnetic-based navigational assembly comprising: (i) a magnetic generator placed adjacent to the body of the patient, and the magnetic generator generates a magnetic field; (ii) at least one magnetic sensor coupled to the first catheter and configured to generate a first signal based on the magnetic field, and the first signal is associated with a three-dimensional space location inside the body of the patient; and (iii) a magnetic navigation module configured to receive and process the first signal, and the magnetic navigation module computes the three-dimensional space location based on the first signal; an impedance-based navigational assembly comprising: (i) a plurality of surface patches attached to the body of the patient; (ii) an impedance navigation module configured to output a plurality of impedance localization signals on the surface patches to generate a multi-axis impedance localization field, and the one or more electrodes of the functional assembly are configured to generate one or more second signals corelated to the position of the associated electrode within the multi-axis impedance localization field, and the one or more second signals each comprise at least a magnitude value and a phase value, and the impedance navigation module further receives and processes the one or more second signals from the one or more electrodes; and a conversion module configured to establish an impedance conversion matrix between the second signals and three-dimensional space locations inside the body of the patient based on the first signal and the physical relationship between the at least one magnetic sensor and the one or more electrodes. The conversion module can be configured to establish a magnetic conversion matrix, and the magnetic navigation module can compute the three-dimensional space location of the at least one magnetic sensor using the magnetic conversion matrix. The impedance conversion matrix can be calibrated by fitting a catheter model template with the measured impedance fields to three-dimensional space locations. The catheter model template can be derived from mathematical equations based on the location of the at least one magnetic sensor and the physically constrained separation distances and/or orientation of the one or more electrodes of the functional assembly. The catheter model template can be determined from a lookup table of previously defined set of physical measurements. The physical measurements can relate to a range of deployment levels of the functional assembly and/or a set of geometric configurations of the functional assembly. The navigation sub-system can be configured to calculate the distance and/or angles between two or more of the at least one magnetic sensors, and the navigation sub-system can be further configured to calculate the position of one or more of the one or more electrodes based on the calculated distance and/or angles and the catheter model template. The navigation sub-system can be configured to assume a nonlinear relationship between the multi-axis impedance localization field and the three-dimensional space within the patient. The navigation sub-system can be configured to divide a volume within the patient into a set of cubic voxel cells, and the cells can establish a relationship between the second signals and the three-dimensional positions of the one or more electrodes. Each of the cubic voxel cells can comprise a length of at least 2 mm and/or a length of no more than 20 mm. The impedance conversion matrix can comprise a conversion matrix for each cell of the set of cubic voxel cells. The navigation sub-system can be further configured to calculate an average error for a conversion matrix of a cell of the set of cubic voxel cells, and to update the conversion matrix if the error is above a threshold. The magnetic navigation module can be configured to determine an optimal twisting angle of the functional assembly. The optimal twisting angle can be determined based on the first signal from the at least one magnetic sensor. The at least one magnetic sensor can comprise at least two magnetic sensors comprising five degree of freedom magnetic sensors. The at least one magnetic sensor can comprise a six degree of freedom magnetic sensor. The first catheter can be at least one of a mapping catheter, an ablation catheter, and/or diagnostic catheter. The catheter device can further comprise at least a second catheter, and the second catheter does not comprise a magnetic sensor, and the impedance conversion matrix can be used to localize the second catheter. The second catheter can comprise at least one of a mapping catheter, an ablation catheter, and/or a diagnostic catheter. Each signal of the plurality of signals can have the same or different frequency. The system can further comprise one or more body surface magnetic sensors configured to be attached to the skin of the patient and to produce a third signal, and the system can be configured to compensate for patient body movement based on the third signal. One or more of the plurality of body surface patches can each comprise one of the one or more body surface magnetic sensors. The third signal can be used to track motion of the torso of the patient relative to the magnetic field generated by the magnetic generator. The system can be further configured to construct a mathematical transformation used to remove components of body motion from localization information. The mathematical transformation can comprise a linear transformation selected from the group consisting of: identity; translation; rotation; scale; shear; and combinations thereof. The transformation can comprise a scale transformation that can be used by the system to compensate for respiratory motion. The plurality of surface patches and/or the impedance localization signals can be configured to avoid null points within an area of interest. The system can be configured to compute data that is provided as if recorded from one or more virtual electrodes and/or one or more virtual magnetic sensors. The navigation sub-system can be configured to determine the expected shape of the functional assembly based on an identified catheter model and the deployment geometry of the functional assembly. The navigational sub-system can be further configured to determine the deployment geometry of the functional assembly based on the first signal from the at least one magnetic sensor. The system can further comprise a force sensor configured to measure the deployment force of the functional assembly, and the navigation sub-system can be further configured to determine the deployment geometry based on the deployment force. The navigation sub-system can be configured to compensate for respiration and/or cardiac artifacts by subtracting a signal reconstructed with orthogonal bases, and the signal represents the artifacts. The navigation sub-system can be further configured to determine if a pattern of respiration and/or cardiac motion has changed, and to update the orthogonal bases if the pattern has changed. The orthogonal bases can be updated within a moving window of time. The navigation sub-system can be further configured to estimate respiration and/or cardiac motion by fitting a periodic or quasiperiodic signal to the first and/or second signals. The navigation sub-system can be configured to compensate for respiration and/or cardiac artifacts using frequency selective filters. The frequency selective filters can remove frequency components higher than 1 Hz.

According to another aspect of the present inventive concepts, a method for localizing a catheter without a magnetic sensor comprises: (a) establishing an impedance conversion matrix between measured impedance fields and three-dimensional space locations inside a body of a patient by: (i) inserting a first catheter into the body of the patient, the first catheter comprises a magnetic sensor and a first electrode configured to form a fiducial pair; (ii) maneuvering the first catheter inside the body of the patient while recording a first set of signals from the magnetic sensor and a second set of signals from the first electrode, the first set of signals correspond to three-dimensional space locations of the magnetic sensor inside the body of the patient; and (iii) establishing a dictionary based on the location of the magnetic sensor and the second set of signals recorded from the first electrode; (b) storing the dictionary; and (c) estimating the position of a second electrode of a second catheter: (i) recording a third set of signals from the second electrode of the second catheter; and (ii) estimating a location of the second electrode of the second catheter based on the third set of signals and the impedance conversion matrix and/or by using an algorithm performed in a global coordinate system defined by the dictionary. The second catheter does not comprise a magnetic sensor, and the second set of signals and the third set of signals comprise impedance-based signals.

In some embodiments, the second catheter comprises an ablation and/or a diagnostic catheter.

In some embodiments, the conversion is used to localize a catheter without magnetic coils.

The technology described herein, along with the attributes and attendant advantages thereof, will best be appreciated and understood in view of the following detailed description taken in conjunction with the accompanying drawings in which representative embodiments are described by way of example.

### INCORPORATION BY REFERENCE

All publications, patents, and patent applications mentioned in this specification are herein incorporated by reference to the same extent as if each individual publication, patent, or patent application was specifically and individually indicated to be incorporated by reference. The content of all publications, patents, and patent applications mentioned in this specification are herein incorporated by reference in their entirety for all purposes.

### BRIEF DESCRIPTION OF THE DRAWINGS

FIG. 1 illustrates a block diagram of an embodiment of a diagnostic mapping system, consistent with the present inventive concepts.

FIG. 1A illustrates a block diagram of another embodiment of a diagnostic mapping system, consistent with the present inventive concepts.

FIG. 2 illustrates a side view of an embodiment of a catheter including a handle and a functional assembly, consistent with the present inventive concepts.

FIG. 3 illustrates a perspective view of the distal portion of an embodiment of a catheter including a basket-like functional assembly, consistent with the present inventive concepts.

FIG. 4 illustrates a side view of the distal portion of an embodiment of a catheter including a basket-like functional assembly, consistent with the present inventive concepts.

FIG. 4A illustrates a cross-sectional view of the functional assembly of FIG. 3, consistent with the present inventive concepts.

FIGS. 5A and 5B illustrate side views of various embodiments of splines of a functional assembly including staggered and aligned electrodes, respectively, consistent with the present inventive concepts.

FIGS. 6A and 6B illustrate anatomic views showing embodiments of patch electrodes placement arrangements, consistent with the present inventive concepts.

FIGS. 7A-D illustrate perspective views of catheters with various embodiments of functional assemblies including electrodes and magnetic sensors, consistent with the present inventive concepts.

FIG. 8 illustrates a flowchart of a method of localizing a catheter, consistent with the present inventive concepts.

FIG. 9 illustrates a flowchart of a method of a process to update a dictionary grid, consistent with the present inventive concepts.

FIG. 10 illustrates a flowchart of a method of estimating electrode locations based on an impedance conversion matrix, consistent with the present inventive concepts.

FIG. 11 illustrates a flowchart of a method of estimating electrode locations with a hybrid algorithm, consistent with the present inventive concepts.

FIG. 12 illustrates a schematic plot of obtaining an optimal twisting angle in a local coordinate system, consistent with the present inventive concepts.

FIG. 13 illustrates a flowchart of a method of compensating for respiration and cardiac motion, consistent with the present inventive concepts.

### DETAILED DESCRIPTION OF THE DRAWINGS

Reference will now be made in detail to the present embodiments of the technology, examples of which are illustrated in the accompanying drawings. Similar reference numbers may be used to refer to similar components. However, the description is not intended to limit the present disclosure to particular embodiments, and it should be construed as including various modifications, equivalents, and/or alternatives of the embodiments described herein.

It will be understood that the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) when used herein, specify the presence of stated features, integers, steps, operations, elements, and/or components, but do not preclude the presence or addition of one or more other features, integers, steps, operations, elements, components, and/or groups thereof.

It will be further understood that, although the terms first, second, third, etc. may be used herein to describe various limitations, elements, components, regions, layers and/or sections, these limitations, elements, components, regions, layers and/or sections should not be limited by these terms. These terms are only used to distinguish one limitation, element, component, region, layer or section from another limitation, element, component, region, layer or section. Thus, a first limitation, element, component, region, layer or section discussed below could be termed a second limitation, element, component, region, layer or section without departing from the teachings of the present application.

It will be further understood that when an element (also referred to as a “component” herein) is described as being “on”, “attached”, “connected” or “coupled” to another element, it can be directly on or above, or connected or coupled to, the other element, or one or more intervening elements can be present. In contrast, when an element is referred to as being “directly on”, “directly attached”, “directly connected” or “directly coupled” to another element, there are no intervening elements present. Other words used to describe the relationship between elements should be interpreted in a like fashion (e.g. “between” versus “directly between,” “adjacent” versus “directly adjacent,” etc.).

As used herein, the terms “operably attached”, “operably connected”, “operatively coupled” and similar terms related to attachment of components shall refer to attachment of two or more components that results in one, two, or more of: electrical attachment; fluid attachment; magnetic attachment; mechanical attachment; optical attachment; sonic attachment; and/or other operable attachment arrangements. The operable attachment of two or more components can facilitate the transmission between the two or more components of: power; signals; electrical energy; fluids or other flowable materials; magnetism; mechanical linkages; light; sound such as ultrasound; and/or other materials and/or components.

It will be further understood that when a first element is referred to as being “in”, “on” and/or “within” a second element, the first element can be positioned: within an internal space of the second element, within a portion of the second element (e.g. within a wall of the second element); positioned on an external and/or internal surface of the second element; and combinations of one or more of these.

As used herein, the term “proximate”, when used to describe proximity of a first component or location to a second component or location, is to be taken to include one or more locations near to the second component or location, as well as locations in, on and/or within the second component or location. For example, a component positioned proximate an anatomical site (e.g. a blood or other fluid delivery location), shall include components positioned near to the anatomical site, as well as components positioned in, on and/or within the anatomical site.

Spatially relative terms, such as “beneath,” “below,” “lower,” “above,” “upper”, “under” and the like may be used to describe an element and/or feature's relationship to another element(s) and/or feature(s) as, for example, illustrated in the figures. It will be further understood that the spatially relative terms are intended to encompass different orientations of the device in use and/or operation in addition to the orientation depicted in the figures. For example, if the device in a figure is turned over, elements described as “below” and/or “beneath” other elements or features would then be oriented “above” the other elements or features. The device can be otherwise oriented (e.g. rotated 90 degrees or at other orientations) and the spatially relative descriptors used herein interpreted accordingly.

The terms “reduce”, “reducing”, “reduction” and the like, where used herein, are to include a reduction in a quantity, including a reduction to zero. Reducing the likelihood of an occurrence shall include prevention of the occurrence. Correspondingly, the terms “prevent”, “preventing”, and “prevention” shall include the acts of “reduce”, “reducing”, and “reduction”, respectively.

The term “and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

The term “one or more”, where used herein can mean one, two, three, four, five, six, seven, eight, nine, ten, or more, up to any number.

The terms “and combinations thereof” and “and combinations of these” can each be used herein after a list of items that are to be included singly or collectively. For example, a component, process, and/or other item selected from the group consisting of: A; B; C; and combinations thereof, shall include a set of one or more components that comprise: one, two, three or more of item A; one, two, three or more of item B; and/or one, two, three, or more of item C.

In this specification, unless explicitly stated otherwise, “and” can mean “or”, and “or” can mean “and”. For example, if a feature is described as having A, B, or C, the feature can have A, B, and C, or any combination of A, B, and C. Similarly, if a feature is described as having A, B, and C, the feature can have only one or two of A, B, or C.

As used herein, when a quantifiable parameter is described as having a value “between” a first value X and a second value Y, it shall include the parameter having a value of: at least X, no more than Y, and/or at least X and no more than Y. For example, a length of between 1 and 10 shall include a length of at least 1 (including values greater than 10), a length of less than 10 (including values less than 1), and/or values greater than 1 and less than 10.

The expression “configured (or set) to” used in the present disclosure may be used interchangeably with, for example, the expressions “suitable for”, “having the capacity to”, “designed to”, “adapted to”, “made to” and “capable of” according to a situation. The expression “configured (or set) to” does not mean only “specifically designed to” in hardware. Alternatively, in some situations, the expression “a device configured to” may mean that the device “can” operate together with another device or component.

As used herein, the terms “about” or “approximately” shall refer to +20% of a stated value.

As used herein, the term “threshold” refers to a maximum level, a minimum level, and/or range of values correlating to a desired or undesired state. In some embodiments, a system parameter is maintained above a minimum threshold, below a maximum threshold, within a threshold range of values, and/or outside a threshold range of values, such as to cause a desired effect (e.g. efficacious therapy) and/or to prevent or otherwise reduce (hereinafter “prevent”) an undesired event (e.g. a device and/or clinical adverse event). In some embodiments, a system parameter is maintained above a first threshold (e.g. above a first temperature threshold to cause a desired therapeutic effect to tissue) and below a second threshold (e.g. below a second temperature threshold to prevent undesired tissue damage). In some embodiments, a threshold value is determined to include a safety margin, such as to account for patient, user, and/or operator variability, system variability, tolerances, and the like. As used herein, “exceeding a threshold” relates to a parameter going above a maximum threshold, below a minimum threshold, within a range of threshold values and/or outside of a range of threshold values.

As described herein, “room pressure” shall mean pressure of the environment surrounding the systems and devices of the present inventive concepts. Positive pressure includes pressure above room pressure or simply a pressure that is greater than another pressure, such as a positive differential pressure across a fluid pathway component such as a valve. Negative pressure includes pressure below room pressure or a pressure that is less than another pressure, such as a negative differential pressure across a fluid component pathway such as a valve. Negative pressure can include a vacuum but does not imply a pressure below a vacuum. As used herein, the term “vacuum” can be used to refer to a full or partial vacuum, or any negative pressure as described hereabove.

The term “diameter” where used herein to describe a non-circular geometry is to be taken as the diameter of a hypothetical circle approximating the geometry being described. For example, when describing a cross section, such as the cross section of a component, the term “diameter” shall be taken to represent the diameter of a hypothetical circle with the same cross sectional area as the cross section of the component being described.

The terms “major axis” and “minor axis” of a component where used herein are the length and diameter, respectively, of the smallest volume hypothetical cylinder which can completely surround the component.

As used herein, the term “functional element” is to be taken to include one or more elements constructed and arranged to perform a function. A functional element can comprise a sensor and/or a transducer. In some embodiments, a functional element is configured to deliver energy. In some embodiments, a functional element is configured to treat tissue (e.g. a functional element configured as a treatment element). Alternatively or additionally, a functional element (e.g. a functional element comprising a sensor) can be configured to record one or more parameters, such as a patient physiologic parameter; a patient anatomical parameter (e.g. a tissue geometry parameter); a patient environment parameter; and/or a system parameter. In some embodiments, a sensor or other functional element is configured to perform a diagnostic function (e.g. to gather data used to perform a diagnosis). In some embodiments, a functional element is configured to perform a therapeutic function (e.g. to deliver therapeutic energy and/or a therapeutic agent). In some embodiments, a functional element comprises one or more elements constructed and arranged to perform a function selected from the group consisting of: deliver energy; extract energy (e.g. to cool a component); deliver a drug or other agent; manipulate a system component or patient tissue; record or otherwise sense a parameter such as a patient physiologic parameter or a system parameter; and combinations of one or more of these. A functional element can comprise a fluid and/or a fluid delivery system. A functional element can comprise a reservoir, such as an expandable balloon or other fluid-maintaining reservoir. A “functional assembly” can comprise an assembly constructed and arranged to perform a function, such as a diagnostic and/or therapeutic function. A functional assembly can comprise an expandable assembly. A functional assembly can comprise one or more functional elements.

The term “transducer” where used herein is to be taken to include any component or combination of components that receives energy or any input, and produces an output. For example, a transducer can include an electrode that receives electrical energy, and distributes the electrical energy to tissue (e.g. based on the size of the electrode). In some configurations, a transducer converts an electrical signal into any output, such as: light (e.g. a transducer comprising a light emitting diode or light bulb), sound (e.g. a transducer comprising a piezo crystal configured to deliver ultrasound energy); pressure (e.g. an applied pressure or force); heat energy; cryogenic energy; chemical energy; mechanical energy (e.g. a transducer comprising a motor or a solenoid); magnetic energy; and/or a different electrical signal (e.g. different than the input signal to the transducer). Alternatively or additionally, a transducer can convert a physical quantity (e.g. variations in a physical quantity) into an electrical signal. A transducer can include any component that delivers energy and/or an agent to tissue, such as a transducer configured to deliver one or more of: electrical energy to tissue (e.g. a transducer comprising one or more electrodes); light energy to tissue (e.g. a transducer comprising a laser, light emitting diode and/or optical component such as a lens or prism); mechanical energy to tissue (e.g. a transducer comprising a tissue manipulating element); sound energy to tissue (e.g. a transducer comprising a piezo crystal); chemical energy; electromagnetic energy; magnetic energy; and combinations of one or more of these.

As used herein, the term “fluid” can refer to a liquid, gas, gel, or any flowable material, such as a material which can be propelled through a lumen and/or opening.

As used herein, the term “material” can refer to a single material, or a combination of two, three, four, or more materials.

As used herein, the term “user interface” can comprise one or more interfaces, each interface comprising one or more components configured to receive an input from a user, “user input device” herein, and/or one or more components configured to provide output to a user, “user output device” herein.

The terms “data” and “information” are used interchangeably herein.

It is appreciated that certain features of the inventive concepts, which are, for clarity, described in the context of separate embodiments, may also be provided in combination in a single embodiment. Conversely, various features of the inventive concepts which are, for brevity, described in the context of a single embodiment, may also be provided separately or in any suitable sub-combination. For example, it will be appreciated that all features set out in any of the claims (whether independent or dependent) can be combined in any given way.

It is to be understood that at least some of the figures and descriptions of the inventive concepts have been simplified to focus on elements that are relevant for a clear understanding of the inventive concepts, while eliminating, for purposes of clarity, other elements that those of ordinary skill in the art will appreciate may also comprise a portion of the inventive concepts. However, because such elements are well known in the art, and because they do not necessarily facilitate a better understanding of the inventive concepts, a description of such elements is not provided herein.

Terms defined in the present disclosure are only used for describing specific embodiments of the present disclosure and are not intended to limit the scope of the present disclosure. Terms provided in singular forms are intended to include plural forms as well, unless the context clearly indicates otherwise. All of the terms used herein, including technical or scientific terms, have the same meanings as those generally understood by an ordinary person skilled in the related art, unless otherwise defined herein. Terms defined in a generally used dictionary should be interpreted as having meanings that are the same as or similar to the contextual meanings of the relevant technology and should not be interpreted as having ideal or exaggerated meanings, unless expressly so defined herein. In some cases, terms defined in the present disclosure should not be interpreted to exclude the embodiments of the present disclosure.

Provided herein are systems, devices, and methods for diagnosing cardiac disease, such as by mapping cardiac electrical activity to identify undesired conduction pathways. A catheter comprising a functional assembly can include one or more electrodes for recording cardiac electrical activity. The catheter can include one or more magnetic sensors for localizing the catheter within the patient. The system can include a console for operating the catheter, where the console comprises a diagnostic module for recording signals from the one or more electrodes and a processing unit for processing the recorded signals.

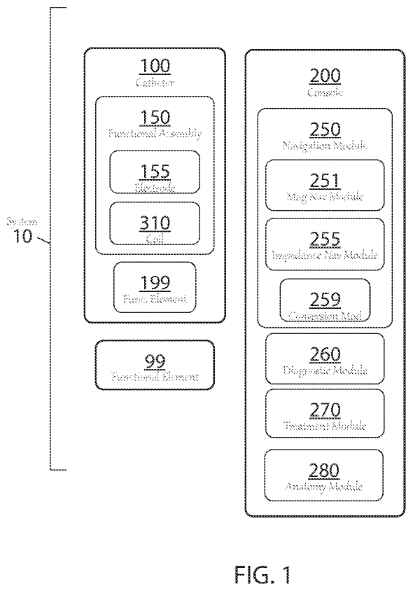

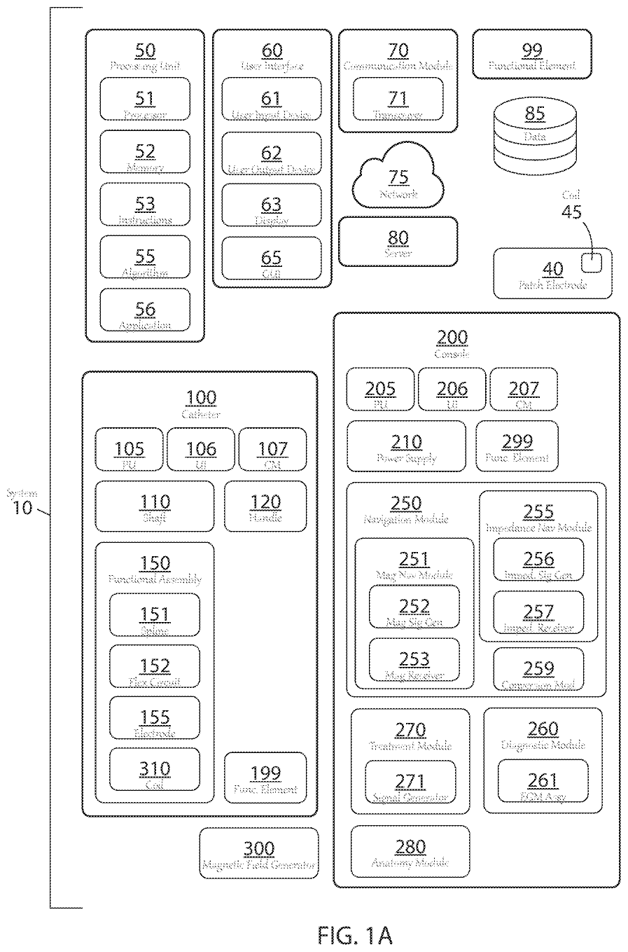

Referring now to FIG. 1, a block diagram of an embodiment of a diagnostic mapping system is illustrated, consistent with the present inventive concepts. System 10 can be configured to map electric magnitudes, morphologies, conduction pathways, physiologic states, and/or other electrical properties or activity within the cardiac tissue of a patient (“electrical activity” herein). System 10 can include one or more catheters, catheter 100 shown, configured to be introduced into a cardiac chamber of the patient. Catheter 100 can include functional assembly 150 comprising at least one or more electrodes, electrode 155 shown. System 10 can include console 200 that is configured to operably attach to catheter 100, such as to record one or more signals from functional assembly 150, to deliver energy to functional assembly 150 (e.g., ablative energy), and/or to facilitate one or more other functions of system 10 as described herein.

Catheter 100 can comprise one or more catheters selected from the group consisting of: a mapping catheter; an ablation catheter; a delivery catheter, such as a right atrial access catheter; a catheter configured to be placed in the coronary sinus (e.g., a CS catheter); and combinations of these. One or more catheters 100 of system 10 can be configured to perform cardiac mapping and ablation, which can include creating a three-dimensional (3D) map of one or more chambers of the patient's heart, and ablating cardiac tissue, such as tissue that is determined (e.g., by the clinician and/or automatically or semiautomatically by system 10) to be a contributing cause of an arrythmia of the patient.

In some embodiments, console 200 includes navigation module 250 that is configured to provide one or more drive signals, and/or to record one or more signals, as described herein. Navigation module 250 can be configured to analyze the recorded signals to provide tracking (or “navigation” or “localization” herein) of at least a portion of one or more catheters 100 and/or other components of system 10 within the patient (e.g., to navigate functional assembly 150 within the cardiac chamber). Navigation module 250 can be configured to execute one, two, or more localization techniques, for example impedance-based localization, magnetic localization, and/or hybrid localization comprising both impedance-based and magnetic localization. Localization of one or more catheters or other devices that are inserted into the patient can be performed to determine the position and/or orientation (“position” herein) of at least a portion of the device (e.g., functional assembly 150 of catheter 100) during a clinical procedure. Navigation module 250 can include magnetic navigation module 251, and/or impedance navigation module 255, each configured to execute magnetic and impedance-based navigation processes, respectively, as described herein. In some embodiments, navigation module 250 includes conversion module 259 that is configured to integrate localization information determined by modules 251 and 255 to execute hybrid localization, as described herein. In some embodiments, catheter 100 includes one or more magnetic navigational sensors, coil 310 shown. Coil 310 can be located on a portion of functional assembly 150, shaft 110, and/or another portion of catheter 100. Coil 310 can include one or more magnetic coils and/or other magnetic field-responsive elements and can be configured to produce one or more signals related to a magnetic field, such as is described herein.

In some embodiments, console 200 includes treatment module 270 that provides one or more electric signals to be delivered to the patient via functional assembly 150, where the electric signals are delivered to treat the tissue. Console 200 can include diagnostic module 260 that records one or more signals (e.g., signals recorded via functional assembly 150) to diagnose the patient, for example to map the cardiac electrical pathways of the patient, as described herein. For example, system 10 can be configured to perform contact and/or non-contact mapping of cardiac electrical pathways. In some embodiments, non-contact mapping (e.g., where electrodes 155 of catheter 100 do not contact the cardiac walls while recording electrical activity) provides increased accuracy (e.g., versus contact mapping) for the mapping of cardiac electrical pathways, such as while the heart is in atrial fibrillation. For example, non-contact mapping can provide simultaneous global mapping of the majority (e.g., an entire) of a cardiac chamber (e.g., the electrical activity of an entire chamber can be mapped near-instantaneously). This simultaneous mapping of an entire chamber can provide increased accuracy when mapping complex, irregular cardiac electrical activity, for example when the heart is in atrial fibrillation.

In some embodiments, console 200 includes anatomy module 280 that is configured to provide a digital anatomic model, for example a 3D model of at least a portion of the patient's heart, such as a chamber of the heart to be mapped by system 10, as described herein. Anatomy module 280 can be configured to import a 3D model, such as a 3D model generated from an imaging device, such as an MRI and/or a CT imaging device. Alternatively, or additionally, anatomy module 280 can be configured to provide signals and/or collect data from one or more devices of system 10 (e.g., catheter 100), such as to generate a 3D anatomic model. For example, catheter 100 can comprise one or more ultrasonic transducers configured to transmit and receive ultrasonic signals, and anatomy module 280 can be configured to generate a 3D model of the anatomy based on the ultrasonic data. In some embodiments, system 10 is configured to localize one or more devices relative to the anatomic model (e.g., when the anatomic model is similarly localized to a 3D coordinate system established by system 10, as described herein). One or more localized devices can be displayed relative to the anatomic model, such as on a display of system 10, as described herein.

System 10 of FIG. 1 can include similar components and can otherwise be of similar construction and arrangement to system 10 described in reference to FIG. 1A and/or other figures described herein.

System 10 can include one or more functional elements, such as functional element 99 shown. One or more components of system 10, such as catheter 100 and/or console 200 can each include a functional element 99, such as when functional elements 199 and/or 299, respectively, each shown, comprise a functional element 99. Singly or collectively, various functional elements described herein can individually or collectively be referred to as “functional element 99”.

Referring additionally to FIG. 1A, a block diagram of another embodiment of a diagnostic mapping system is illustrated, consistent with the present inventive concepts. In some embodiments, one or more components of system 10 of FIG. 1A are of similar construction and arrangement to the similar components described in reference to FIG. 1 herein.

System 10 can include one or more data processing modules, processing unit 50 shown, that can be configured to perform and/or facilitate one or more of the functions of system 10 described herein. For example, processing unit 50 can perform and/or facilitate one or more processes, data collections, data analyses, data transfers, signal processing functions, energy deliveries, monitoring of one or more patient parameters, and/or other functions of system 10 (“functions of system 10” or “system functions” herein). Processing unit 50 can comprise one or more electronic elements, electronic assemblies, and/or other electronic components, such as components selected from the group consisting of: microprocessors; microcontrollers; state machines; memory storage components; analog-to-digital converters; rectification circuitry; amplifiers, filters, and/or other signal conditioners; sensor interface circuitry; transducer interface circuitry; and combinations of one, two, or more of these. For example, processing unit 50 can include at least one processor and at least one memory storage component, such as processor 51 and memory 52, each shown. Memory 52 can be coupled to processor 51, and memory 52 can store one or more sets of computer instructions, instructions 53 shown. Instructions 53 can comprise instructions used by processor 51 to perform one or more algorithms of system 10. For example, system 10 can comprise one or more algorithms, algorithm 55 shown, that are performed by processor 51. Additionally, or alternatively, instructions 53 can comprise instructions for running one or more applications of system 10, for example application 56 shown. Processing unit 50 can be configured to “run” application 56, such that application 56 can initiate, modify, stop, and/or coordinate the performance of various functions of console 200 and/or of system 10. In some embodiments, application 56 is configured to receive input from a user of system 10 (e.g., a clinician, nurse, or other operator of system 10), for example via a user interface (e.g., user interface 60 described herein). In some embodiments, algorithm 55 can comprise one or more machine learning, neural net, and/or other artificial intelligence algorithms (“AI algorithm” herein). All or a portion of one or more processing units 50 can be integrated into one, two, or more of the various components of system 10, such as console 200, a server (e.g., server 80 described herein), and/or other components of system 10.

System 10 can include one or more user interfaces, user interface 60 shown. User interface 60 can provide and/or receive information to and/or from a user of the system (e.g., a clinician and/or other user of system 10). User interface 60 can include one or more user input components and/or output components. For example, user interface 60 can comprise a keyboard, mouse, touchscreen, and/or other human interface or other input component (e.g., as described herein), user input device 61. In some embodiments, user interface 60 can comprise a speaker, indicator light, haptic transducer and/or other human interface or other output component (e.g., as described herein), user output device 62. In some embodiments, user output device 62 comprises a video output component, such as display 63 shown. Display 63 can comprise a touchscreen display, for example when user input device 61 and user output device 62 collectively comprise display 63. In some embodiments, processing unit 50 is configured to provide an interactive graphical interface, GUI 65, such as a graphical user interface provided by application 56. GUI 65 can be displayed (e.g., displayed to a user of system 10) via display 63. In some embodiments, user interface 60 and/or GUI 65 comprise a virtual reality and/or augmented reality interface. One or more components of system 10 can comprise one or more portions of a user interface 60, such as catheter 100, console 200, and/or other components of system 10 described herein.

System 10 can include one or more communication modules, communication module 70 shown. One or more devices of system 10 can comprise one or more portions of a communication module 70, such as catheter 100, console 200, and/or other components of system 10 described herein. Communication module 70 can be configured to provide communication (e.g., transfer commands, delivery information, patient information, and/or other data) between two or more components of system 10, such as via wired and/or wireless communication. For example, communication module 70 can include one or more transmitters and/or receivers, transceiver 71 shown. Transceiver 71 can comprise a wireless transceiver, such as a Bluetooth transceiver, a Near Field Communication (NFC) transceiver, a Wi-Fi transceiver, a cellular transceiver, a satellite-connected transceiver, and/or other short-range and/or long-range wireless transceiver. A wireless connection can include a short-range wireless connection, such as an NFC connection and/or a Bluetooth low energy (BLE) connection. In some embodiments, communication module 70 is configured to transfer data via an acoustic signal, such as an acoustic signal that is outside of the auditory range of the user. In some embodiments, communication module 70 is configured to communicate via one or more wired and/or wireless networks, such as network 75 shown. Network 75 can include a wireless network, such as a cellular network, LAN, WAN, VPN, the Internet, and/or other wireless network connecting two or more devices. In some embodiments, network 75 comprises a wired network, and/or a network including wired and wireless devices.

Communication module 70 can be configured to transfer data between at least a first component of system 10 and at least a second component of system 10, as described herein. In some embodiments, the first component of system 10 comprises catheter 100. The second component can comprise another component of system 10, for example console 200.

In some embodiments, system 10 includes one or more servers, server 80 shown, that can be configured to provide data storage and/or data processing for the providers of system 10 (e.g., the manufacturer and/or distributor of system 10) and/or the users or patients of system 10. As used herein, data processing can refer to the receiving of data, processing of data, transmission of data (e.g., transmitting the results of data processing), and/or the storage of data. Server 80 can comprise one or more processing units 50. Additionally, or alternatively, server 80 can include one or more data storage units for storing data collected by system 10, data 85 shown. In some embodiments, server 80 is configured to process data from various users of system 10, for example when the provider of system 10 maintains one or more servers 80 configured to process data for each (and/or a subset) of the users of system 10 (e.g., each of the clinicians, patients, and/or other users of system 10). Server 80 can comprise an “off-site” server (e.g., remotely located from the users of system 10), such as a server owned, maintained, and/or otherwise provided by the provider of system 10. Alternatively, or additionally, server 80 can comprise a cloud-based server.

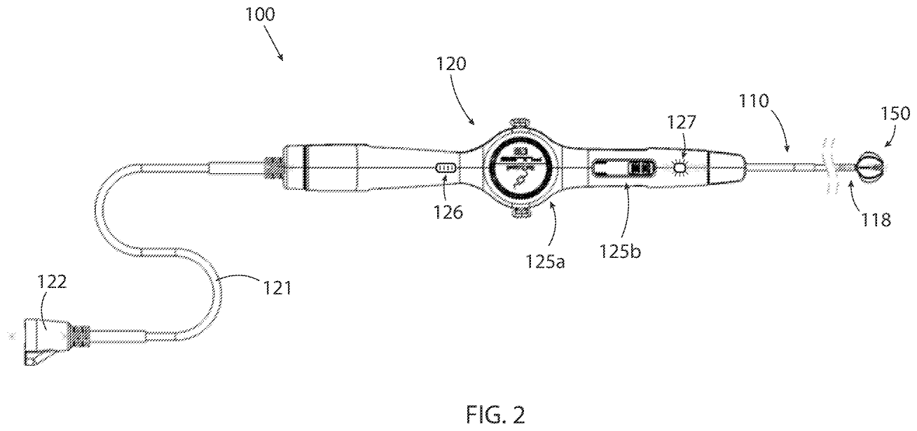

In some embodiments, catheter 100 includes at least a portion of processing unit 50, at least a portion of user interface 60, and/or at least a portion of communication module 70, such as when catheter 100 comprises processing unit 105, user interface 106, and/or communication module 107, respectively, each shown. In some embodiments, console 200 includes at least a portion of processing unit 50, at least a portion of user interface 60, and/or at least a portion of communication module 70, such as when console 200 comprises processing unit 205, user interface 206, and/or communication module 207, respectively, each shown. In some embodiments, processing unit 205 is configured to perform one or more functions of navigation module 250, diagnostic module 260, treatment module 270, and/or anatomy module 280, such as are described herein. For example, processing unit 205 can be configured to process data received from catheter 100 by navigation module 250 and/or diagnostic module 260, such as to localize a device and/or to map cardiac electrical activity, as described herein. Algorithm 55 can be executed by processing unit 50 to perform one or more of the functions described in reference to the various modules of console 200. Console 200 can be configured to facilitate interaction between the clinician and system 10, for example providing an interface for procedure planning, execution, and/or review. Display 63 of user interface 60 can be coupled to console 200 and provide a visual output (e.g., GUI 65) for displaying real-time data, imaging, and/or other information needed by the physician during the procedure. In some embodiments, console 200 comprises two or more separate console units (e.g., two or more units each comprising a housing surrounding various components described herein). For example, console 200 can comprise a workstation, such as a workstation comprising a user interface, such as user interface 206, and a separate console (e.g., a separate unit operably attached to the workstation unit) housing one or more modules, such as navigation module 250, diagnostic module 260, treatment module 270, and/or anatomy module 280. In some embodiments, a workstation unit of console 200 comprises a user device such as a tablet or desktop computer that provides an interface for controlling a separate unit of console 200, such as a separate unit comprising signal and/or power generators and/or various data acquisition modules (e.g., components of modules 250, 260, 270, and/or 280, described herein). In some embodiments, two or more separate units of console 200 and/or two or more other components of system 10 (e.g., console 200 and catheter 100) are connected via one or more cables, such as cable 121 described in reference to FIG. 2 and otherwise herein.

Catheter 100 can include an elongated body, shaft 110 shown, extending from a user control portion, handle 120 shown. Functional assembly 150 can be located on a distal portion of shaft 110, for example when functional assembly 150 comprises an expandable array that is located at the distal end of shaft 110. An embodiment of functional assembly 150 comprising an expandable array is described in reference to FIG. 2 and otherwise herein.

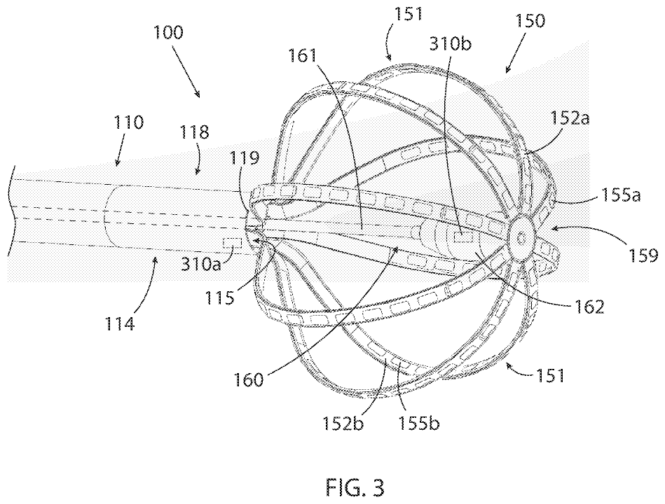

Functional assembly 150 can include an expandable array, such as an expandable array of one or more flexible struts, splines 151 shown, such as two, four, six, eight, ten, or twelve splines 151. Functional assembly 150 can include one or more flexible circuit portions, flex circuit 152 shown, such as a flex circuit 152 located on each side (e.g., the “inside” of each spline, toward the center of functional assembly 150, and the “outside” of each spline, away from the center of functional assembly 150) of each spline 151. Each flex circuit 152 can include one or more electrodes 155, where an electrode 155 positioned on an inside flex circuit 152 is oriented toward the center of functional assembly 150, and an electrode 155 positioned on an outside flex circuit 152 is oriented away from the center of functional assembly 150, for example as described in reference to FIG. 3 and otherwise herein.

Functional assembly 150 can comprise a high density of electrodes 155 and/or a high number of electrodes 155. For example, functional assembly 150 can comprise at least four splines 151, such as at least six, eight, ten, or twelve splines 151. Each spline 151 can include at least three electrodes 155 per side (e.g., inward-facing and outward-facing, as described herein), such as at least six, eight, ten, twelve, fourteen, or sixteen electrodes 155 per side.

Splines 151 can comprise a material selected from the group consisting of: nickel-titanium alloy; stainless steel; polyethylenimine (PEI); polyimide; and combinations of these. Each spline 151 can comprise a flexible and/or elastic member, such as an elastic ribbon (e.g., a flat sheet), such as a nickel-titanium (e.g., Nitinol) ribbon. Each spline can comprise a length of at least 15 mm, and/or a length of no more than 35 mm. Each flex circuit 152 can be operably attached to a portion (e.g., side) of an associated spline 151. For example, flex circuits 152 can be laminated, bonded, and/or otherwise attached to splines 151. Electrodes 155 can comprise a material selected from the group consisting of: gold; platinum; platinum-iridium; iridium oxide; PDOT conductive polymer; titanium nitride; graphene; a precious metal alloy; and combinations of these. In some embodiments, each electrode 155 comprises a coating, such as a coating selected from the group consisting of: a gold coating; a coating configured to decrease the input impedance of the electrode; a PDOT coating; an iridium oxide coating; a titanium nitride coating; an oxide coating; and combinations of these. Each electrode 155 can comprise a thickness of at least 0.01 μm and/or no more than 10 μm. Each electrode 155 can comprise a surface area of at least 0.3 mm² and/or no more than 5.0 mm².

Each electrode 155 of functional assembly 150 can be operably attached to a portion of a flex circuit 152, for example to an electrical trace of flex circuit 152 that is operably (e.g., electrically) connectable to console 200, such via one or more electrical conductors (e.g., one or more wires or flex circuits), and one or more connectors configured to operably attach catheter 100 to console 200, such as cable 121 including connector 122 as described in reference to FIG. 2 and otherwise herein. In some embodiments, flex circuit 152 includes one or more vias connecting one or more electrical components on various layers of the circuit. In some embodiments, one or more vias connecting electrodes 155 to electrical traces of flex circuit 152 can comprise a coating, such as a gold or other coating.

As described herein, catheter 100 can include one or more magnetic navigational sensors, such as coil 310, shown. Coil 310 can be located on a portion of functional assembly 150, such as to provide a signal related to the location, orientation, and/or geometric configuration (e.g., whether in an expanded or collapsed geometric configuration) of functional assembly 150, as described herein. Coil 310 can comprise a multi-degree of freedom sensor, such as a three, four, five, and/or six degree-of-freedom (DOF) sensor.

Console 200 can include power supply 210 that provides electrical power to the various components of console 200. In some embodiments, power supply 210 is configured to provide patient isolation, for example when console 200 is configured to receive electrical power from an outlet or other external power source, and power supply 210 includes patient protection circuitry configured to protect the patient from the external power source. This isolation can protect the patient from potential electrical hazards, ensuring that catheter 100 can safely operate within the patient's body.

Diagnostic module 260 can include a cardiac signals processing assembly, EGM assembly 261. EGM assembly 261 can be configured to receive and record signals from electrodes 155 of catheter 100, and to process the recorded signals to diagnose and/or otherwise evaluate the patient, such as by mapping the cardiac electrical pathways of the patient.

Treatment module 270 can be configured to provide electrical energy to catheter 100 to be delivered to the tissue of the patient (e.g., via one or more electrodes 155), such as to ablate and/or otherwise treat the tissue. Treatment module 270 can include one or more signal generators, such as signal generator 271 shown, configured to generate the electrical signal to be delivered to the tissue. Signal generator 271 can be configured to generate an RF signal, such as a signal configured to thermally ablate tissue, and/or an electroporation signal, such as a signal configured to reversibly and/or irreversibly electroporate the tissue.

Magnetic navigation module 251 can include a signal generator, magnetic signal generator 252, that generates one or more magnetic drive signals, and/or a signal receiver, magnetic signal receiver 253, that receives and records signals from one or more magnetic sensors, such as coil 310 of catheter 100. System 10 can include magnetic field generator 300 that receives the drive signal from magnetic signal generator 252 and generates one or more magnetic localization fields proximate the patient (e.g., to generate a magnetic field encompassing at least a cardiac chamber within which functional assembly 150 is to be inserted). Each coil 310 can generate a signal (e.g., a signal that is recorded by magnetic signal receiver 253) related to the position of the coil within the magnetic localization field generated by magnetic field generator 300. For example, each coil 310 can receive a magnetic signal (e.g., sense the magnetic field generated by magnetic field generator 300) and produce an electrical signal based on the received magnetic signal.

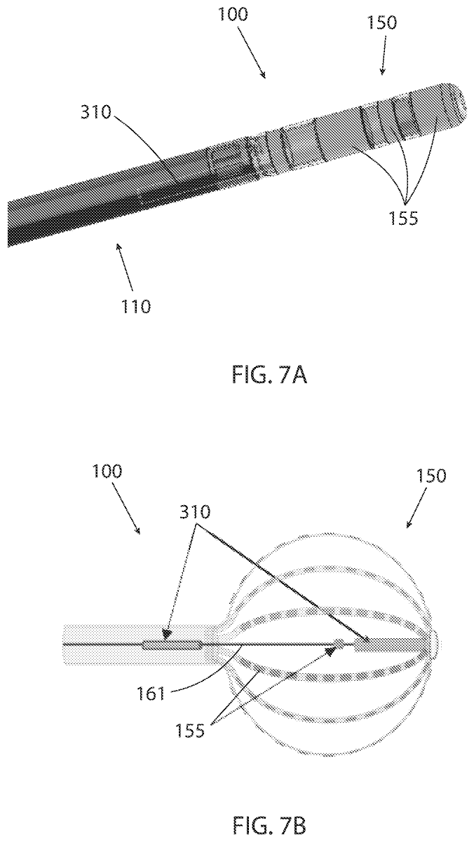

Impedance navigation module 255 can include a signal generator, impedance signal generator 256, that generates one or more electric drive signals, and/or a signal receiver, impedance signal receiver 257, that receives and records signals from one or more electrodes, such as electrode 155 of functional assembly 150. System 10 can include one or more external patient electrodes, patch electrodes 40. Impedance signal generator 256 can be configured to deliver the generated electric drive signals between sets of two or more patch electrodes 40, such as to establish a multi-axis impedance localization field within the patient. In some embodiments, a multi-axis impedance localization field can be configured as described in reference to FIG. 6A and/or FIG. 6B herein. In some embodiments, signals provided by impedance signal generator 256 comprise different frequencies, for example when signals comprising different frequencies are delivered between different pairs of patch electrodes 40, such that each axis of the multi-axis impedance localization field comprises a signal (e.g., current flowing through the patient between the patch pairs) with a different frequency.

In some embodiments, navigation module 250 defines a 3D coordinate system surrounding the patient within which the various devices of system 10 can be localized. The magnetic localization field generated by magnetic field generator 300 can be correlated to the 3D coordinate system, such that the position of a magnetic sensor that is determined relative to the magnetic localization field via magnetic localization can be correlated to a position within the 3D coordinate system. Additionally, or alternatively, the multi-axis impedance localization field generated by signals delivered between patch electrodes 40 can be correlated to the 3D coordinate system, such that the position of an electrode that is determined relative to the impedance localization field via impedance-based localization can be correlated to a position within the 3D coordinate system. In some embodiments, a first conversion matrix (“magnetic conversion matrix” herein) can be used by conversion module 259 of navigation module 250 to convert position data determined by magnetic localization to position data relative to the 3D coordinate system. Additionally, or alternatively, a second conversion matrix (“impedance conversion matrix” herein) can be used by conversion module 259 of navigation module 250 to convert position data determined by impedance-based localization to position data relative to the 3D coordinate system, as described herein.

As described herein, at least a portion of catheter 100, such as functional assembly 150, comprising one or more coils 310 and/or electrodes 155, can be inserted into the patient, and navigation module 250 can be configured to localize the inserted portion of catheter 100 within the patient. Navigation module 250 can establish a 3D coordinate system for localizing catheter 100 within the patient. In some embodiments, magnetic signal receiver 253 is configured to record one or more signals from the one or more coils 310 of catheter 100, where the signals are related to the position of the coils 310 within the magnetic field generated by magnetic field generator 300. Conversion module 259 can be configured to localize coils 310 within the 3D coordinate system based on the signals recorded by magnetic signal receiver 253 (e.g., by establishing the magnetic conversion matrix, as described herein). Impedance signal receiver 257 can be configured to record one or more signals from one or more electrodes 155 of catheter 100, where the signals are related to the position of the electrodes 155 within an electrical field generated by signals provided by impedance signal generator 256 to patch electrodes 40, as described herein. In some embodiments, signals recorded by impedance signal receiver 257 comprises phase values, magnitude values, and/or both, that are related to the multi-axis impedance field described herein. In some embodiments, patch electrode 40 includes one or more magnetic sensors, for example coil 45 shown, such as a sensor that is configured to produce a signal related to the location of patch electrode 40 within the 3D coordinate system based on magnetic localization signals, such that patch electrode 40 can be localized to the 3D coordinate system, as described herein.

One or more coils 310 and electrodes 155 of catheter 100 can form fiducial pairs, where the physical relationship between the elements of the fiducial pairs have a known physical spatial relationship. Conversion module 259 can be configured to establish the impedance conversion matrix based on the known relationship of the fiducial pairs, the localization data of the coils 310 of the fiducial pairs, and the signals recorded by impedance signal receiver 257 from the electrodes 155 of the fiducial pairs. In some embodiments, impedance signal receiver 257 is configured to record signals from one or more electrodes 155 not fiducially paired with a coil 310 (e.g., unpaired electrodes 155 of a catheter including one or more fiducial pairs, and/or electrodes 155 of another catheter 155 that does not comprise any coils 310). Conversion module 259 can be configured to localize one or more electrodes 155 within the 3D coordinate system based on the signals recorded by impedance signal receiver 257 and the impedance conversion matrix.

In some embodiments, conversion module 259 is configured to calibrate the magnetic conversion matrix and/or the impedance conversion matrix (singly or collectively “conversion matrices” herein). Conversion module 259 can be configured to calibrate a conversion matrix based on fitting the localized position of one or more elements (e.g., coils 310 and/or electrodes 155) to a known physical spatial relationship of the elements of catheter 100 (e.g., based on a known model of catheter 100).

In some embodiments, one or more magnetic sensors (e.g., coils 45) can be attached to the patient (e.g., positioned on the skin of the patient), and signals from these sensors can be used by system 10 to compensate for patient body motion during a procedure, such as motion relative to magnetic field generator 300. For example, two or more magnetic sensors (e.g., patch electrodes 40 comprising coils 45) can be attached to the patient. These sensors can be positioned at the same location as patch electrodes 40 (e.g., coils 45 that are integral to patch electrodes 40), and/or can be positioned at other locations on the body that are separate from patch electrodes 40 (e.g., non-electrode patient patches comprising coil 45). Signals recorded from coils 45 can be used to track the motion of the torso of the patient relative to the 3D coordinate system and can provide information to an algorithm (e.g., algorithm 55) that compensates for undesired, artifactual components of motion that are imposed upon navigation of one or more catheters within the body. The location and/or orientation of coils 45 can be recorded at the start of the procedure for reference. During the procedure, the current location and/or orientation of coils 45 can be used to construct a mathematical transformation. The transformation can be linear or non-linear, and its inverse transformation can be used to remove components of body motion from the magnetic-based and impedance-based catheter localization information. This method of body motion compensation can be applied on navigation of one or more catheters or treatment or diagnostic devices of system 10 that are localized within the body. The linear transformation can be any of “identity” (no motion), “translation” (movement in space without rotation), “rotation” (change of orientation in space), “scale” (change of volume), “shear” (sliding and stretching motion along one direction), or any combination of these. In some embodiments, the linear transformation simply amounts to a rigid body motion (a combination of a “translation” and a “rotation”). More accurate nonlinear transformations can also be constructed when three or more coils 45 are used. In some embodiments, compensation for respiratory motion can be performed using the “scale” transformation type. Linear affine transformation for different motion types is provided by the following equations.

$${A}_{identity} = [ \begin{matrix}1 & 0 & 0 & 0 \\ 0 & 1 & 0 & 0 \\ 0 & 0 & 1 & 0 \\ 0 & 0 & 0 & 1\end{matrix} ]$$

$${A}_{translation} = [ \begin{matrix}1 & 0 & 0 & {T}_{x} \\ 0 & 1 & 0 & {T}_{y} \\ 0 & 0 & 1 & {T}_{z} \\ 0 & 0 & 0 & 1\end{matrix} ]$$

$${A}_{rotation} = [ \begin{matrix}{R}_{xx} & {R}_{xy} & {R}_{xz} & 0 \\ {R}_{yx} & {R}_{yy} & {R}_{yz} & 0 \\ {R}_{zx} & {R}_{zy} & {R}_{zz} & 0 \\ 0 & 0 & 0 & 1\end{matrix} ]$$

$${A}_{scale} = [ \begin{matrix}{S}_{x} & 0 & 0 & 0 \\ 0 & {S}_{y} & 0 & 0 \\ 0 & 0 & {S}_{z} & 0 \\ 0 & 0 & 0 & 1\end{matrix} ]$$

$${A}_{shear} = [ \begin{matrix}1 & {γ}_{xy} & {γ}_{xz} & 0 \\ 0 & 1 & {γ}_{yz} & 0 \\ 0 & 0 & 1 & 0 \\ 0 & 0 & 0 & 1\end{matrix} ]$$

Referring now to FIG. 2, a side view of an embodiment of a catheter including a handle and a functional assembly is illustrated, consistent with the present inventive concepts. Catheter 100 and/or other components of system 10 described in FIG. 2 can be of similar construction and arrangement as the similar components described in reference to FIG. 1 and otherwise herein. Catheter 100 can include a user control portion, handle 120. Shaft 110 can extend distally from handle 120, with functional assembly 150 positioned proximate a distal portion, distal portion 118 shown, of shaft 110. Catheter 100 can include one or more interconnects, cable 121, including one or more conduits, such as one or more electrical, fluid carrying, optical, mechanical linkages, and/or other conduits configured to operably attach catheter 100 to console 200 and/or other components of system 10. Cable 121 can include connector 122 that removably attaches to a component of system 10 (e.g., console 200), and creates one or more connections, such as one or more electrical, fluid, optical, mechanical, and/or other operable connections between catheter 100 and the connected component. In some embodiments, cable 121 provides a fluid connection between console 200 and catheter 100, for example when catheter 100 is configured to provide irrigation via one or more lumens extending through shaft 110 to one or more irrigation ports proximate functional assembly 150, not shown but known to those skilled in the art.

Handle 120 can include one or more user controls (e.g., one or more controls of user interface 60, such as user input device 61). For example, handle 120 can include one or more mechanical controls that allow the user to mechanically manipulate a portion of catheter 100, such as steering control 125a and/or deployment control 125b, each shown. Steering control 125a can operably attach to one or more steering cables (not shown) that are arranged to manipulate the articulation of the distal portion 118 of shaft 110. Deployment control 125b can operably attach to one or more linkages (not shown but described herein) that are arranged to manipulate the geometry of functional assembly 150, for example to transition functional assembly 150 from a linear (compacted) geometry, to a radially expanded geometry (e.g., when functional assembly 150 comprises an expandable basket arrangement, as described herein). Additionally, or alternatively, handle 120 can include one or more user input controls (e.g., user inputs configured to provide an input signal to console 200), user input 126 shown. User input 126 can be configured to provide an input signal, such as a signal configured to start and/or stop a process of system 10 described herein, for example to record data (e.g., during a cardiac mapping process) and/or to deliver energy (e.g., during an ablation process). Handle 120 can include one or more outputs (e.g., one or more outputs of user interface 60, such as user output device 62). For example, handle 120 can include output 127, such as an output device comprising a visual indicator, audible indicator, haptic indicator, and/or other output device, such as described herein.

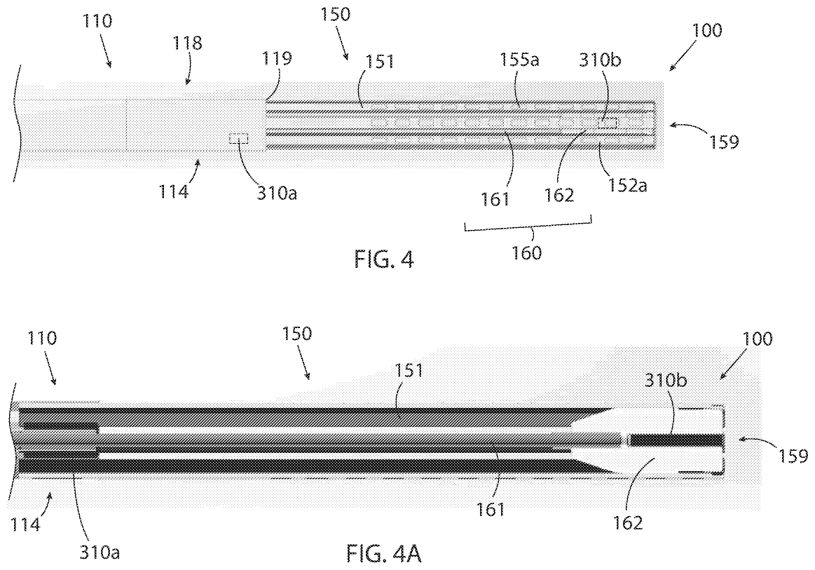

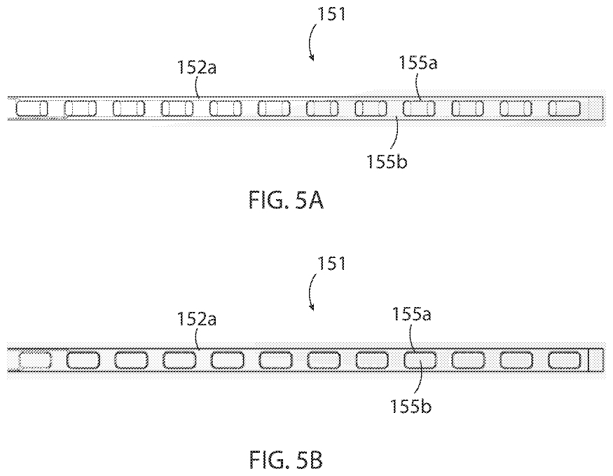

Referring now to FIG. 3, a perspective view of the distal portion of an embodiment of a catheter including a basket-like functional assembly is illustrated, consistent with the present inventive concepts. Catheter 100 and/or other components of system 10 described in FIG. 3 can be of similar construction and arrangement as the similar components described in reference to FIG. 1 and otherwise herein. In the embodiment of FIG. 3, functional assembly 150 comprises a basket-like array, shown in an expanded geometry. FIG. 3 shows the distal portion of catheter 100, including distal portion 118 of shaft 110, and functional assembly 150. Shaft 110 terminates at distal end 119, and can include one or more lumens extending therethrough, such as lumen 115 extending proximally from distal end 119 through shaft 110. Distal portion 118 of shaft 110 can include a coupling mechanism, coupler housing 114, that connects functional assembly 150 to shaft 110, as described herein. Catheter 100 can include control assembly 160 that is constructed and arranged to control the geometry of functional assembly 150. Control assembly 160 can include one or more linkages, such as puller tube 161 shown, and a distal hub assembly, housing 162, that attaches to and/or forms the distal portion of functional assembly 150. In some embodiments, for example as shown in FIG. 3, the proximal portion of each spline 151 is fixedly attached to coupler housing 114, and the distal portion of each spline 151 is fixedly attached to housing 162. Puller tube 161 can be advanced and/or retracted through lumen 115, such as to adjust the distance between housing 162 and distal end 119 of shaft 110, thereby adjusting the geometry of functional assembly 150 (e.g., by straightening and/or deflecting each spline 151 to transition between a linear geometry and an expanded geometry). Puller tube 161 can be operably attached to a user control, such as deployment control 125b described in reference to FIG. 2 herein. Alternatively, or additionally, puller tube 161 can be operably attached to a linear actuator or other electromechanical actuator, such as when catheter 100 is configured to be automatically and/or semi-automatically controlled (e.g., robotically controlled). Functional assembly 150 is shown in an expanded geometry in FIG. 3. FIG. 4, described herein, shows functional assembly 150 in a linear geometry. Functional assembly 150 can be configured to expand to a diameter of up to at least 12 mm, such as at least 16 mm and/or up to no more than 35 mm, such as no more than 24 mm.

Functional assembly 150 can comprise distal surface 159, as shown. In some embodiments, distal surface 159 comprises the distal most end of catheter 100, for example when no part of shaft 110, puller tube 161, and/or housing 162 extends beyond distal surface 159 (e.g., catheter 100 comprises a “nose-less” catheter). For example, the distal ends of splines 151 can be contiguous with distal surface 159, as shown.

Catheter 100 can include one or more coils 310 configured to enable magnetic localization of functional assembly 150 and/or other portions of catheter 100, as described herein. In some embodiments, catheter 100 includes at least a first coil 310a located proximate coupler housing 114 (e.g., at a fixed location relative to distal end 119 of shaft 110), and a second coil 310b located on or within housing 162, each shown. System 10 can be configured to localize each of coils 310a,b and to determine the deployment status of functional assembly 150 based on the determined distance between the coils.

Each spline 151 can include (e.g., positioned thereon) a set of one or more “outward-facing” electrodes (electrodes facing away from the center of functional assembly 150), electrodes 155a, and/or a set of one or more “inward-facing” electrodes (electrodes facing toward the center of functional assembly 150), electrodes 155b, each shown. Each electrode 155 can be electrically coupled to one or more conduits, such as one or more traces of a circuit assembly, such as flex circuit 152 described herein. In some embodiments, flex circuit 152 comprises a flexible circuit board construction, such as flexible substrate with electrical traces connecting various components of the circuit (e.g., electrodes 155 located thereon). Alternatively, or additionally, flex circuit 152 can include one or more wires that electrically connect to various electrical components of functional assembly 150 (and/or catheter 100), such as individual wires without a “circuit board” like substrate. Functional assembly 150 can comprise one or more discrete flex circuits, where each flex circuit 152 can comprise all or a portion of a discrete flex circuit, for example when a single flex circuit comprise two or more portions, each of which are configured to be positioned relative to a portion of functional assembly 150. Various discrete flex circuits can be interconnected by one or more connectors, wires, or other electrical conduits.

Each spline 151 can include (e.g., positioned thereon) a flex circuit 152a positioned on the outside of the spline, and/or a flex circuit 152b positioned on the inside of the spline (e.g., such that each outward-facing electrode 155a is positioned on a flex circuit 152a, and each inward-facing electrode 155b is positioned on a flex circuit 152b, as shown). In some embodiments, each flex circuit 152a and 152b of each spline 151 comprises a single discrete flex circuit that is “folded” such that a first portion of the flex circuit comprises the outward flex circuit 152a, and a second portion of the flex circuit comprises the inward flex circuit 152b. Alternatively, or additionally, each of the flex circuits 152a,b of each spline 151 can comprise discrete flex circuits, such as discrete flex circuits that are interconnected as described herein. In some embodiments, functional assembly 150 comprises a first discrete flex circuit comprising each outward flex circuit 152a (e.g., a single discrete flex circuit comprising multiple elongate portions that are each positioned along the outside of an associated spline 151), and/or a second discrete flex circuit comprising each inward flex circuit 152b (e.g., a single discrete flex circuit comprising multiple elongate portions that are each positioned along the inside of an associated spline 151). In some embodiments, a single discrete flex circuit comprises multiple elongate portions each comprising a flex circuit 152a or 152b, where the single flex circuit is configured to be folded and/or otherwise manipulated (e.g., in a manufacturing process), to align with the respective inside and/or outside of splines 151.

Referring now to FIG. 4, a side view of the distal portion of an embodiment of a catheter including a basket-like functional assembly is illustrated, consistent with the present inventive concepts. Catheter 100 and/or other components of system 10 described in FIG. 4 can be of similar construction and arrangement as the similar components described in reference to FIG. 1 and otherwise herein. In the embodiment of FIG. 4, functional assembly 150 comprises a basket-like array, shown in a collapsed (“linear”) geometry. FIG. 4 shows functional assembly 150 extending from distal end 119 of shaft 110. Distal portion 118 of shaft 110 includes coupler housing 114, as described herein. Puller tube 161 is shown in an extended position (extended distally from shaft 110), such that housing 162 of control assembly 160 is positioned away from coupler housing 114, extending splines 151 into the linear geometry shown. Coils 310a,b are shown located within coupler housing 114 and housing 162, as described herein. In the geometry shown, the distance between coils 310a,b is greater than the relative distance between the coils in the expanded geometry of functional assembly 150 shown in FIG. 3. Each spline 151 can include flex circuits 152 (outward flex circuit 152a shown), and/or electrodes 155 (outward-facing electrodes 155a shown). Distal surface 159 of functional assembly 150 comprises the distal most portion of catheter 100, as shown and described herein.

Referring additionally to FIG. 4A, a cross-sectional view of the functional assembly of FIG. 3 is illustrated, consistent with the present inventive concepts. Coil 310a is shown positioned within coupler housing 114. Coil 310a can comprise a hollow structure, as shown, such that puller tube 161 can slidingly extend through coil 310a. Coil 310b is shown positioned within housing 162. The distal end of each spline 151 can be fixedly attached to the distal end of housing 162, forming distal surface 159, as shown. The proximal end of each spline 151 can extend into and be fixedly attached within coupler housing 114, as shown.

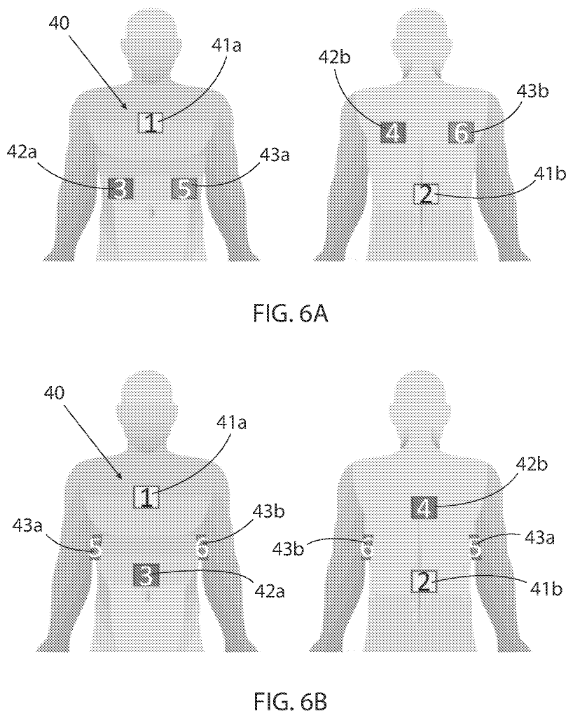

Referring now to FIGS. 5A and 5B, side views of various embodiments of splines of a functional assembly including staggered and aligned electrodes, respectively, are illustrated, consistent with the present inventive concepts. Spline 151 and/or other components of system 10 described in FIGS. 5A and 5B can be of similar construction and arrangement as the similar components described in reference to FIG. 1 and otherwise herein. FIGS. 5A and 5B show the outward-facing sides of various embodiments of spline 151, with flex circuit 152a (shown) positioned on the outward surface, and flex circuit 152b (not shown) positioned on the inward surface, as described herein. Outward-facing electrodes 155a are shown in solid lines, and inward-facing electrodes 155b (positioned on the opposite of the side shown of spline 151) are shown dashed.

In some embodiments, as shown in FIG. 5A, outward-facing electrodes 155a can be axially offset (e.g., along the length of spline 151) from inward-facing electrodes 155b. Alternatively, as shown in FIG. 5B, outward-facing electrodes 155a can be axially aligned (e.g. along the length of spline 151) with inward-facing electrodes 155b. In some embodiments, the alignment of electrodes 155a,b varies between two or more splines 151 of functional assembly 150. In some embodiments, the number of outward-facing electrodes 155a matches and/or varies from the number of inward-facing electrodes 155b.

Referring now to FIGS. 6A and 6B, anatomic views showing embodiments of patch electrodes placement arrangements are illustrated, consistent with the present inventive concepts. Patch electrodes 40 and/or other components of system 10 described in FIGS. 6A and 6B can be of similar construction and arrangement as the similar components described in reference to FIG. 1 and otherwise herein. System 10 can include one or more sets (e.g. “pairs”) of two or more patch electrodes 40, such as a first pair, electrodes 41a,b, a second pair, 42a,b, and/or a third pair 43a,b, as shown in FIGS. 6A and 6B. Impedance signal generator 256 of impedance navigation module 255, not shown but described herein, can be configured to provide an electrical signal between each electrode of a pair of patch electrodes 40, such as to generate an electrical impedance field between the electrodes. Signals comprising varying electrical properties (e.g., signal frequencies) can be provided between different pairs of patch electrodes 40, such as to create a multi-axis impedance localization field. Impedance navigation module 255 can receive and process signals from one or more electrodes located on a patient inserted device (e.g., electrodes 155 of catheter 100), where the signals are related to the multi-axis impedance localization field and are processed to localize the patient inserted device.

The placement and alignment of each pair of patch electrodes 40 can be configured based on procedural needs and/or the patient's physique (e.g., the patient's body size and shape). FIGS. 6A and 6B show two non-limiting embodiments of patch electrode 40 placement configurations. FIG. 6A shows the pair of patient patch electrodes 41a,b positioned centrally on the upper chest and lower back, respectively, of the patient, defining a first axis of the impedance electrical field. The pair of patch electrodes 42a,b are shown positioned on the lower right of the front of the torso and upper left of the back, respectively, of the patient, defining a second axis of the impedance electrical field. The pair of electrodes 43a,b are shown positioned on the lower left of the front of the torso and the upper right of the back, respectively, of the patient, defining a third axis of the impedance electrical field. FIG. 6B shows the pair of patch electrodes 41a,b similarly positioned centrally on the upper chest and lower back, respectively, of the patient, defining a first axis of the impedance electrical field. The pair of patch electrodes 42a,b are shown positioned centrally on the lower torso and upper back, respectively, of the patient, defining a second axis of the impedance electrical field. The pair of patch electrodes 43a,b, are shown positioned on the right and left sides of the torso, respectively, of the patient, defining a third axis of the impedance electrical field. In some embodiments, patch electrodes 40 are positioned, and/or the signals provided are configured, such that the physical locations of zero amplitude (e.g., the null points) of the composite impedance electrical field are located proximate the heart of the patient, but outside of a region of interest (e.g., proximate but outside of a heart chamber), such as to avoid null points (e.g., locations where a the localization signal comprises zero amplitude) within the region of interest. Alternatively, or additionally, the physical location of zero-amplitude of the field for one or more axes can be configured away from the heart of the patient.

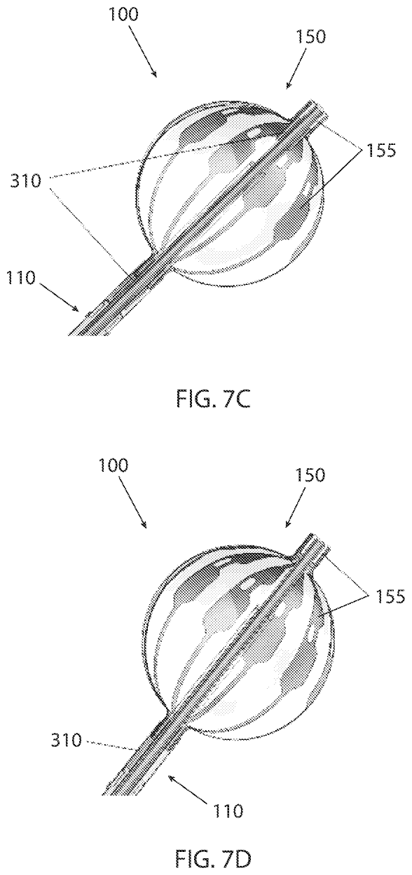

Referring now to FIGS. 7A-D, perspective views of catheters with various embodiments of functional assemblies including electrodes and magnetic sensors are illustrated, consistent with the present inventive concepts. Catheters 100 and/or other components of system 10 described in FIGS. 7A-D can be of similar construction and arrangement as the similar components described in reference to FIG. 1 and otherwise herein. FIGS. 7A-D illustrate various embodiments of catheter 100 including shaft 110 and functional assembly 150, including one or more electrodes 155 and/or coils 310. FIG. 7A shows a linear embodiment of catheter 100. FIGS. 7B-D show various embodiments of expandable functional assemblies 150.

FIG. 7A shows an embodiment of catheter 100 with functional assembly 150 positioned on a distal portion of shaft 110. Electrodes 155 can comprise ring and/or tip electrodes, as shown, positioned on the distal portion of shaft 110. Coil 310 can be positioned within shaft 110, proximate functional assembly 150.

FIG. 7B shows an embodiment of catheter 100 with functional assembly 150 comprising an expandable basket array of electrodes 155. Catheter 100 includes puller tube 161 configured to advance and/or retract from shaft 110 to collapse and/or expand, respectively, functional assembly 150. One or more electrodes 155 and/or coils 310 can be positioned on puller tube 161, as shown.

FIGS. 7C and 7D show embodiments of catheter 100 with functional assembly 150 comprising an expandable balloon with an array of electrodes 155 positioned thereon. One or more coils 310 can be positioned within a distal portion of shaft 110 and/or functional assembly 150.

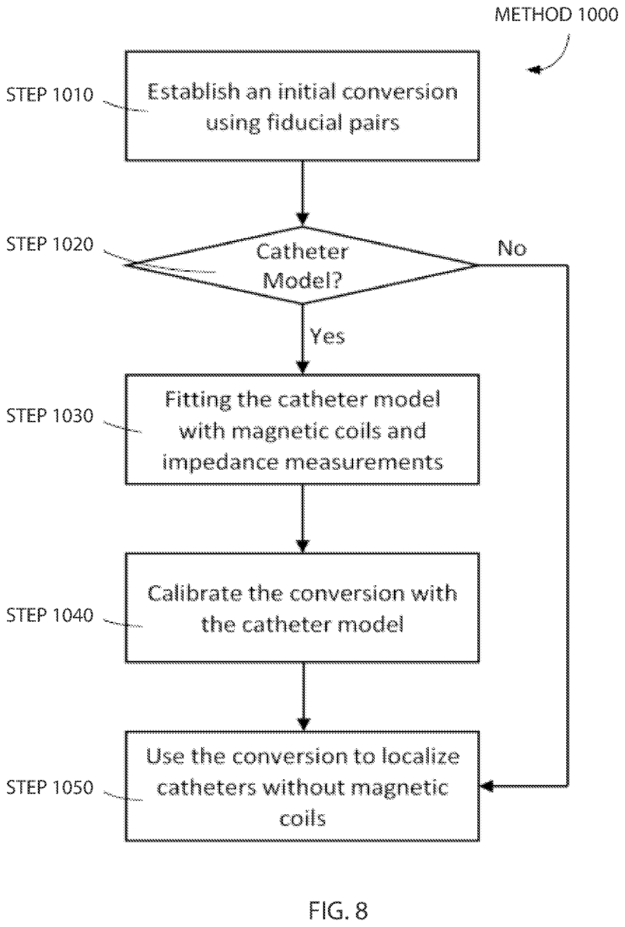

Referring now to FIG. 8, a flowchart of a method of localizing a catheter is illustrated, consistent with the present inventive concepts. Method 1000 of FIG. 8 can be performed using the components of system 10 described in reference to FIG. 1 and otherwise herein. Method 1000 comprises a method of hybrid navigation, including establishing an impedance conversion matrix, calibrating the impedance conversion matrix, and using the impedance conversion matrix to localize one or more devices without magnetic sensors relative to the magnetic localization field and/or the 3D coordinate system established by navigation module 250 described herein. In some embodiments, the magnetic localization field comprises a relatively uniform and/or relatively consistent magnetic field that comprises a direct correlation to the 3D coordinate system established by navigation module 250. The multi-axis impedance localization field can comprise an impedance field that varies throughout different portions of the patient, and/or a field that varies over time, for example based on patient motion, breathing, and/or other perturbations that may cause the field to vary (spatially and/or temporally) and the localization of one or more electrodes to be non-uniform. In Step 1010 of Method 1000, conversion module 259 can be configured to calculate an impedance conversion matrix that is configured to convert impedance-based localization data to locations within the 3D coordinate system, where the impedance conversion matrix is based on impedance-based and magnetic localization data recorded from one or more “fiducial pairs” of electrodes 155 and coils 310 and is configured to compensate for non-uniformity of the multi-axis impedance localization field. In some embodiments, system 10 is configured to compute data that is provided as if recorded from one or more “virtual electrodes” 155 and/or “virtual coils” 310. Virtual electrodes can provide impedance-based localization data that is computed by system 10 based on impedance-based data that is recorded from one or more physical electrodes 155 of catheter 100 and provided as if recorded from the location of the virtual electrode. Virtual coils can provide magnetic localization data that is computed by system 10 based on data that is recorded from one or more physical coils 310 of catheter 100 and provided as if recorded from the virtual coil. Fiducial pairs of electrodes 155 and coils 310 can comprise physical and/or virtual electrodes and/or coils.

In some embodiments, a coil 310 and an electrode 155 (e.g., physical and/or virtual coils and/or electrodes) form a fiducial pair when the distance between the two elements is fixed. In some embodiments, the portion of catheter 100 between the elements of a fiducial pair is rigid, or the portion can be configured to articulate (e.g., bend) in a known and/or predictable manner. Magnetic navigation information recorded from a coil 310 of a fiducial pair, and impedance-based navigation information recorded from a corresponding electrode 155 of the fiducial pair can be processed by console 200 (e.g., by conversion module 259 of navigation module 250), in conjunction with the known physical relationship between the coil and electrode, to calculate one or more impedance conversion matrices that correlate the impedance navigation system to the magnetic navigation system (e.g., and the 3D coordinate system).

The impedance conversion matrix can be calculated based on the localized position of each coil 310 within the 3D coordinate system, the known physical relationship between the fiducial pairs of coils 310 and electrodes 155, and the impedance-based localization data recorded from each electrode 155 (e.g., based on data recorded from one or more fiducial pairs, as described herein). Using the impedance conversion matrix, the impedance-based localization information of other electrodes 155 (e.g., electrodes 155 not fiducially paired with a physical coil 310 and/or a virtual coil, as described in reference to Step 1050) can be converted to position information within the 3D coordinate system.

In Step 1020, navigation module 250 can be configured to determine if catheter 100 comprises a catheter with a known physical configuration (e.g., the physical configuration of coils 310 and electrodes 155 of catheter 100). For example, system 10 can be configured to identify the configuration of catheter 100 based on information provided by the clinician (e.g., the clinician can indicate the configuration of catheter 100 being used), and/or by electronic and/or other automatic and/or semi-automatic identification of catheter 100 (e.g., when catheter 100 comprises an RFID or other identifier configured to provide configuration information to system 10). In some embodiments, navigation module 250 is configured to identify the configuration of catheter 100 based on the recorded localization information of one or more coils 310 of catheter 100. For example, navigation module 250 can comprise a library of various catheter model templates, each template comprising information related to the physical configuration of each catheter (e.g., the physical configuration of the structure of each catheter, and/or the physical relationship between the coils 310 and electrodes 155 of each catheter). Based on the determined locations of the localized magnetic coils 310, if a predefined standard catheter model is identified by navigation module 250 (e.g., from the library of catheter model templates), the template can be used to refine the position information of electrodes 155 that is computed based on the impedance conversion matrix and the impedance-based localization information of electrodes 155, as described in reference to Steps 1030 and 1040. If a known catheter model template is not identified, Method 1000 continues with Step 1050, described herebelow. In Step 1030, the identified catheter model template can be fit (e.g., scaled) based on the localized locations of coils 310 of catheter 100. In some embodiments, the catheter model template is derived from mathematical equations based on the locations of coils 310 and the physically constrained separation distances and orientation of the electrodes. For example, splines and/or other linear arrangements of electrodes can be fitted by offset, rotation, least-squares, bilinear, bicubic, Bezier, and/or other fitting formulas. Alternatively, or additionally, the catheter model template can be determined from a look-up table containing a previously defined set of physical measurements taken from a catheter when it is set at a range of deployment levels (e.g., in deployment steps of 1% or 10% or 25% from fully collapsed to fully opened) and/or into different shapes, as defined by a “phantom curve or surface” that envelopes the splines and/or other configurations of electrodes distributed on the catheter. Once the catheter model template is fit (e.g., scaled) to a catheter 100 that is being localized based on the magnetic localization information from coils 310, the position information converted from the impedance-based localization information of electrodes 155 of catheter 100 can be compared to the catheter model template. In Step 1040, the differences between the fitted electrode locations and computed electrode locations are then used to calibrate the impedance conversion matrix and/or to adjust the position information of electrodes 155 of catheter 100.

In Step 1050, the impedance measurements from electrodes 155 of a catheter 100 (e.g., a catheter 100 with or without coils 310) can be converted, using the impedance conversion matrix, to localize the electrodes 155 within the 3D coordinate system. Additionally, or alternatively, one or more catheters 100 comprising one or more coils 310 can be localized in Step 1050. If in Step 1020, a catheter model was identified, the calibrated impedance conversion matrix (e.g., the matrix calibrated in Step 1040) can be used in Step 1050. If no catheter model was identified in Step 1020, the initial (e.g., uncalibrated) impedance conversion matrix can be used in Step 1050.

In some embodiments, to estimate the relationship between the multi-axis impedance localization fields and the 3D space in the heart chamber (e.g., the 3D coordinate system generated by navigation module 250), Method 1000 assumes ‘local’ linearity of the impedance field. This is inspired by the fact that locally (i.e., in the vicinity of any point in space) the electric field is essentially uniform, and therefore the relationship between the impedance values and location can be assumed to be linear. Alternatively, or additionally, Method 1000 can assume a nonlinear relationship between the multi-axis impedance localization fields and the 3D space in the heart chamber (e.g., the 3D coordinate system generated by navigation module 250). This is to cope with the varying impedance field (e.g., the impedance field close to the heart walls or the impedance field affected by external sources).

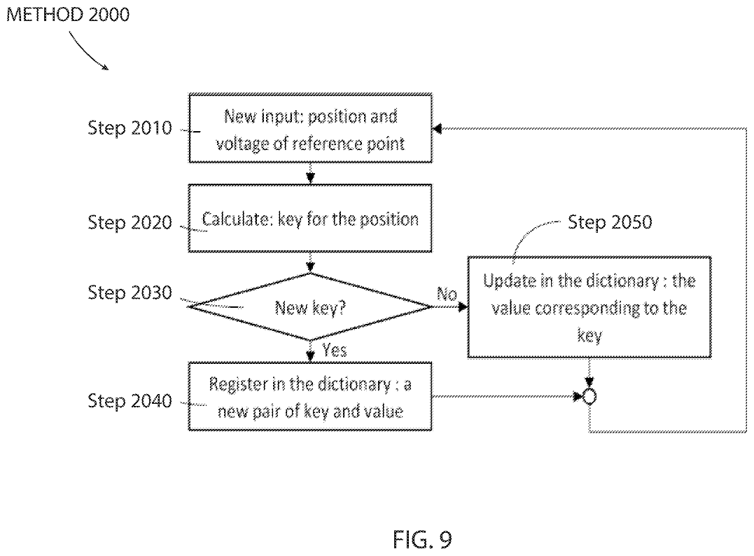

While catheter 100 including one or more fiducial pairs of coils 310 and electrodes 155 is maneuvered within the heart chamber, magnetic localization data and impedance-based localization data from each of the fiducial pairs can be recorded and used to establish a dictionary, for example as described in reference to FIG. 9 herein. In some embodiments, impedance-based localization data can include recorded voltages and/or currents.

An example of a method of establishing a linear relationship between impedance-based localization information (e.g., recorded voltages) and 3D positions based on data recorded from one or more fiducial pairs within a scanned volume (e.g., relative to the 3D coordinate system) is given as follows. The scanned volume can be divided into a set of cubic voxel cells. In some embodiments, the length of the voxel cell's side can comprise a length of at least 2 mm and/or a length of no more than 20 mm. The 3D positions of the fiducial pairs are converted to the indices for the voxel grid by dividing the predefined grid size for each axis (x, y, z). The grid size can be uniform across different axes and/or the voxels can comprise a different grid size for different axes. For example:

$$i = ⌊ x / {g}_{x} ⌋ ; j = ⌊ y / {g}_{y} ⌋ ; k = ⌊ z / {g}_{z} ⌋ \qquad ( 1 )$$

where i, j, k are indices for grid cells and gₓ, g_{y}, g_{z} are grid sizes for three axes.
For the multi-axis impedance localization field inside each grid cell, it can be assumed that the field is uniform and the relationship between the voltage and location in 3D space can be represented by:

$$[ \begin{matrix}{v}_{x} \\ {v}_{y} \\ {v}_{z}\end{matrix} ] = [ \begin{matrix}{u}_{1} & {e}_{11} & {e}_{12} & {e}_{13} \\ {u}_{2} & {e}_{21} & {e}_{22} & {e}_{23} \\ {u}_{3} & {e}_{31} & {e}_{32} & {e}_{33}\end{matrix} ] [ \begin{matrix}1 \\ x \\ y \\ z\end{matrix} ] \qquad ( 2 )$$

The matrix

$$A = [ \begin{matrix}{u}_{1} & {e}_{11} & {e}_{12} & {e}_{13} \\ {u}_{2} & {e}_{21} & {e}_{22} & {e}_{23} \\ {u}_{3} & {e}_{31} & {e}_{32} & {e}_{33}\end{matrix} ]$$

comprises the scaling matrix (e.g., the impedance conversion matrix) that is estimated by conversion module 259 from N number of fiducial pairs

$${\{ ( {x}_{i} , {y}_{i} , {z}_{i} ) , ( {v}_{xi} , {v}_{y i} , {v}_{zi} ) \}}_{i = 1 , … , N} .$$

Equation (2) can be rewritten as:

$$[ \begin{matrix}{v}_{x 1} & {v}_{y 1} & {v}_{z 1} \\ ⋮ & ⋮ & ⋮ \\ {v}_{xN} & {v}_{yN} & {v}_{zN}\end{matrix} ] = [ \begin{matrix}1 & {x}_{1} & {y}_{1} & {z}_{1} \\ & & ⋮ & \\ 1 & {x}_{N} & {y}_{N} & {z}_{N}\end{matrix} ] [ \begin{matrix}{u}_{1} & {u}_{2} & {u}_{3} \\ {e}_{11} & {e}_{21} & {e}_{31} \\ {e}_{12} & {e}_{22} & {e}_{32} \\ {e}_{13} & {e}_{23} & {e}_{33}\end{matrix} ] \qquad ( 3 )$$

### Let

$$P = [ \begin{matrix}1 & {x}_{1} & {y}_{1} & {z}_{1} \\ & & ⋮ & \\ 1 & {x}_{N} & {y}_{N} & {z}_{N}\end{matrix} ] ; V = [ \begin{matrix}{v}_{x 1} & {v}_{y 1} & {v}_{z 1} \\ ⋮ & ⋮ & ⋮ \\ {v}_{xN} & {v}_{yN} & {v}_{zN}\end{matrix} ] and A = [ \begin{matrix}{u}_{1} & {u}_{2} & {u}_{3} \\ {e}_{11} & {e}_{21} & {e}_{31} \\ {e}_{12} & {e}_{22} & {e}_{32} \\ {e}_{13} & {e}_{23} & {e}_{33}\end{matrix} ] .$$

The scaling matrix can be computed using a regularization method, such as the Tikhonov regularization:

$$A = {( {P}^{T} P + λ I )}^{- 1} {P}^{T} V \qquad ( 4 )$$

where λ comprises the regularization parameter to regularize the effects from the measurement noise.

Referring now to FIG. 9, a flowchart of a method of a process to update a dictionary grid is illustrated, consistent with the present inventive concepts. Method 2000 of FIG. 9 can be performed using the components of system 10 described in reference to FIG. 1 and otherwise herein. In some embodiments, impedance-based localization information, magnetic based localization information, and/or other localization information related to one or more grid cells of a voxel grid, such as a voxel grid described in reference to FIG. 8 herein, can be stored in a dictionary type data structure. A dictionary data structure can be used to efficiently store information related to various local grid cells. An embodiment of an implementation of a dictionary data structure is presented in Table 1.

TABLE 1
Dictionary

| List of keys | List of values |
|---|---|
| Key_i: | Value_i: |
| i, j, k index of | Position of the grid cell center; |
| the grid cell. | Scaling matrix of the linear impedance field in |
| | the grid cell; |
| | List of visited positions by the reference point |
| | in the grid cell; |
| | List of corresponding impedance values; |
| | Number of visited positions in the grid cell. |

Method 2000 of FIG. 9 comprises a method of updating a dictionary data structure. In Step 2010, a new input is received by navigation module 250, comprising data related to a fiducial point (e.g., within a voxel of a grid cell). The data can comprise magnetic localization data, impedance-based localization data, and/or other data recorded from a recording element (e.g., coil 310 and/or electrode 155) of catheter 100. In Step 2020, after a new input related fiducial pair is observed, a ‘key’ is generated by navigation module 250 based on position of the fiducial pair. In Step 2030, navigation module 250 determines if the key was previously added to the list of keys in the dictionary, or if the key is new to the dictionary. In Step 2040, if the ‘key’ is not in the dictionary, a ‘value’ is initialized based on the magnetic based localization data and impedance based localization data of the input, and the pair {key, value} is registered into the dictionary. In Step 2050, if the ‘key’ is already in the dictionary, the corresponding ‘value’ is updated based on the magnetic based localization data and impedance-based localization data of the input.

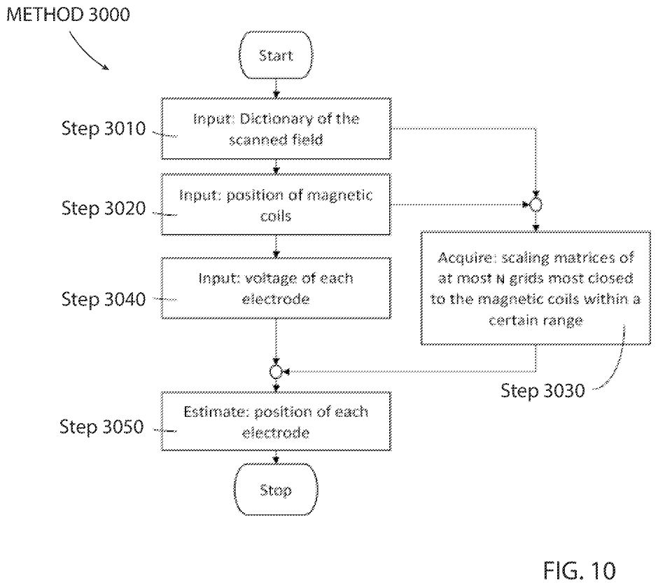

Referring now to FIG. 10, a flowchart of a method of estimating electrode locations based on an impedance conversion matrix is illustrated, consistent with the present inventive concepts. Method 3000 of FIG. 10 can be performed using the components of system 10 described in reference to FIG. 1 and otherwise herein. In some embodiments, system 10 comprises an algorithm (e.g., algorithm 55 described herein), for example a “z-loc” algorithm that is configured to estimate the position of one or more electrodes (e.g., electrodes 155) relative to the 3D coordinate system established by navigation module 250, based on impedance-based localization data.

The z-loc algorithm can provide an initial impedance conversion matrix to estimate the position of each electrode 155 of catheter 100 based on measured impedance values (e.g., impedance-based localization information) recorded from each electrode 155. Method 3000 comprises a method of the z-loc algorithm to estimate the position of each electrode. The z-loc algorithm can be performed to localize one or more impedance-based elements (e.g., electrodes 155) relative to the 3D coordinate system defined by navigation module 250, as described herein. The 3D coordinate system can be correlated to the magnetic-based navigation system, also as described herein. In Step 3010, the z-loc algorithm receives an input comprising impedance conversion matrix information, for example information stored in a dictionary data structure, such as described in reference to FIGS. 8 and 9 and otherwise herein. In Step 3020, the z-loc algorithm receives an input comprising magnetic localization information, such as information related to the position of one or more coils 310 (e.g., the localized position of one more coils 310 relative to the 3D coordinate system established by navigation module 250). In Step 3030, the z-loc algorithm determines one or more impedance conversion matrices, such as one or more scaling matrices as described herein. The z-loc algorithm can determine impedance conversion matrices (e.g., via a lookup of previously calculated matrices and/or by calculating a matrix) for one or more voxel grids surrounding the position of each of coils 310 for which the position was input to the z-loc algorithm. For example, when a dictionary is provided, the z-loc algorithm can perform a search of the dictionary for existing impedance conversion matrices correlated to the grid cells within a certain range of the position of each coil 310. In some embodiments, at most N voxel grid cells closest to the position of each coil 310 are selected from the search results, for example where N is equal to four, six, eight, or ten. In Step 3040, the z-loc algorithm receives an input comprising impedance-based localization information (e.g., voltage data) from one or more electrodes 155. In Step 3050, the positions of all electrodes relative to the 3D coordinate system established by navigation module 250 can be calculated with the impedance conversion matrices (e.g., the identified scaling matrices) associated with the selected grid cells, for example by solving a system of equations, as described in reference to FIG. 8 and otherwise herein.

In some embodiments, the z-loc algorithm comprises one or more biases and/or assumptions. For example, the z-loc algorithm can assume each electrode 155 of catheter 100 and/or the center of catheter 100 (e.g., the center of functional assembly 150 of catheter 100) are in a uniformly distributed impedance field. Additionally, or alternatively, the z-loc algorithm can assume the impedance fields of nearby voxel grid cells do not change significantly compared to the impedance field at the location of the catheter. These two assumptions are known to be true when the impedance-based localization field is uniform and are expected to be good enough for slowly varying impedance-based localization fields. When the impedance-based localization field varies significantly over a short distance, the accuracy is expected to decrease, and for such cases finer grids can be used. Additionally, or alternatively, the z-loc algorithm can assume each electrode 155 of catheter 100 and/or the center of catheter 100 (e.g., the center of functional assembly 150 of catheter 100) are in a nonlinear field that follows a quadratic or higher-order form.

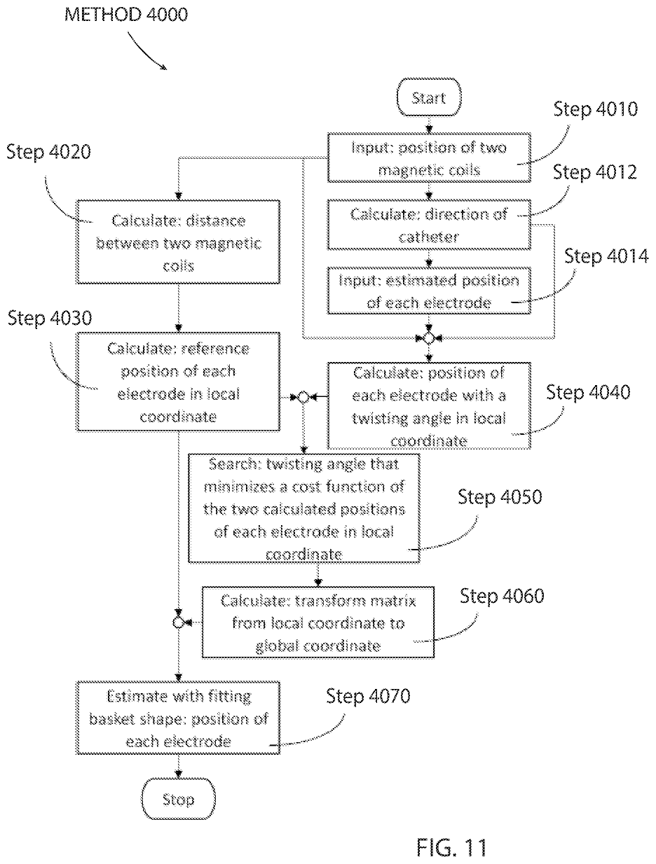

Referring now to FIG. 11, a flowchart of a method of estimating electrode locations with a hybrid algorithm is illustrated, consistent with the present inventive concepts. Method 4000 of FIG. 11 can be performed using the components of system 10 described in reference to FIG. 1 and otherwise herein. In some embodiments, system 10 comprises an algorithm (e.g., algorithm 55 described herein), for example an “h-loc” algorithm that is configured to estimate the position of one or more electrodes (e.g., electrodes 155) relative to the 3D coordinate system established by navigation module 250, based on impedance-based localization data, magnetic localization data, and/or known catheter configurations, as described herein. The h-loc algorithm can provide an improved estimation of the position of one or more electrodes 155 of catheter 100 based on one or more initial estimations calculated by the z-loc algorithm that is described in reference to FIG. 10 and otherwise herein.

In some embodiments, catheter 100 includes one or more magnetic sensors, coils 310 described herein, and/or one or more electrical sensors, electrodes 155 described herein. Catheter 100 can include two or more coils 310 positioned proximate functional assembly 150, for example as described in reference to FIG. 1 and otherwise herein. Two or more coils 310 of catheter 100 can comprise similar or dissimilar configurations, for example two coils 310 of a catheter 100 comprising a basket-shaped functional assembly 150 can comprise two 5-DOF (degree of freedom) coils 310, or one 5-DOF and one 6-DOF coil 310. In some embodiments, a coil 310 can comprise two or more coils or sets of coils, such as two or more multi-degree of freedom coils, for example two 6-DOF coils (e.g., such that coil 310 provides one or more redundant degrees of freedom, or up to 12 degrees of freedom). In some embodiments, a first coil 310 is positioned on a first end of functional assembly 150 and a second coil 310 is located on a second end of functional assembly 150, such that localization of the two coils can be analyzed to determine the length and/or orientation of functional assembly 150, as described herebelow.

In Step 4010, the h-loc algorithm receives magnetic localization information related to two or more coils 310 of catheter 100 (e.g., coils positioned on first and second ends of functional assembly 150). In Step 4012, the h-loc algorithm can calculate the direction and/or orientation of functional assembly 150 relative to the 3D coordinate system of navigation module 250 based on the magnetic localization information. In Step 4014, the h-loc algorithm receives estimated localized positions of one or more electrodes 155, such as estimations calculated by the z-loc algorithm, as described herein.

In Step 4020, based on the magnetic localization information (e.g., the positions of coils 310 relative to the 3D coordinate system established by navigation module 250), the distance and/or angles between coils 310 are calculated. In Step 4030, this calculated distance and/or angle can be used to calculate the positions of one or more electrodes 155 of catheter 100 . . . within a local coordinate system based on a standard catheter model template. As described herein, a standard catheter model template can be mathematically modeled, physically measured from a physically existing catheter, or both.

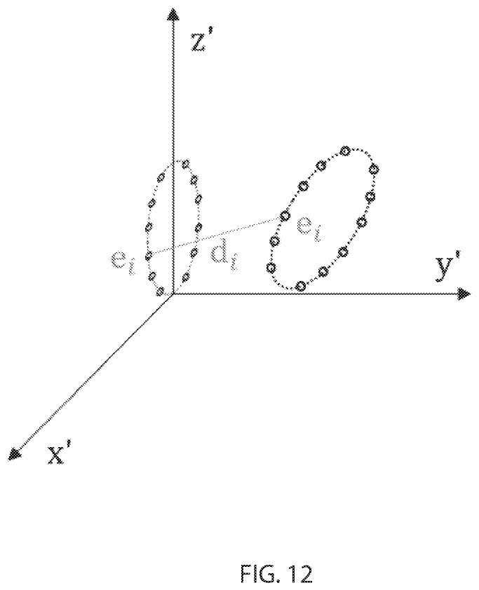

In Step 4040, the h-loc algorithm can calculate any twist (e.g., the twisting angle) of functional assembly 150. For example, when coils 310 comprise multi-degree of freedom sensors, such as one, two, or more 5-DOF coils 310 and/or at least one 6-DOF coil 310 of functional assembly 150, the twisting angle can be computed from the magnetic localization data recorded from coils 310. Alternatively, or additionally, if functional assembly 150 comprises two 5-DOF coils 310 each positioned on opposite ends of functional assembly 150, an optimal twisting angle can be estimated based on the magnetic localization data recorded from coils 310 as well as the estimated locations of electrodes 155 computed by the z-loc algorithm, as described herein. A schematic plot for obtaining this optimal twisting angle is shown and described in reference to FIG. 12 herein. The estimated positions of one or more electrodes 155 of functional assembly 150 are transformed to a local coordinate system using the z-loc algorithm. In Step 4050, an optimal twisting angle can be computed, such as computed by minimizing a cost function E di, where di is the distance between the location of each electrode eᵢ in the local coordinate system, and the location of the same electrode in the reference untwisted catheter based on the identified catheter model template (e.g., as determined in Step 4040). In Step 4060, based on the calculated optimal twisting angle, the measured catheter direction, and the measured positions of the magnetic coils, the h-loc algorithm can calculate a conversion matrix, such as a transform matrix, that is configured to correlate the positions of electrodes 155 in the local coordinate system established in Method 4000, to the global 3D coordinate system established by navigation module 250. In Step 4070, the estimated positions of each electrode 155 can be further improved based on fitting the shape of the localized electrodes 155 to the shape of functional assembly 150 from the identified catheter model template within the global 3D coordinate system (e.g., after the positions are transformed from the local coordinate system to the global 3D coordinate system in Step 4060).

In some embodiments, the expected shape of functional assembly 150 (e.g., the expected spatial relationship between the localized elements of functional assembly 150) can be calculated based on an identified catheter model, and the deployment geometry of functional assembly 150. The deployment geometry can be calculated based on the length and/or angle of the functional assembly 150 (e.g., based on magnetic localization information from coils 310 positioned on opposite ends of functional assembly 150) and/or based on the amount of mechanical push and/or pull force applied by a deployment mechanism of catheter 100, such as puller tube 161 of catheter 100 (e.g., as described in reference to FIG. 3 and otherwise herein). In some embodiments, the push and/or pull force applied by a deployment mechanism of catheter 100 can be measured from the handle (e.g., handle 120) of catheter 100. In some embodiments, the deployment force and the calculated length of functional assembly 150 are used in conjunction to determine the expected shape of functional assembly 150. The h-loc algorithm can use estimated positions of one or more electrodes 155 provided by the z-loc algorithm to estimate the twisting angle, and use the twisting angle to transform the calculated shape of functional assembly 150. The h-loc algorithm can provide a more accurate estimation of the localized positions of electrodes 155 relative to the 3D coordinate system of navigation module 250, even though the estimation is based on the less accurate z-loc algorithm.

Referring now to FIG. 12, a schematic plot of obtaining an optimal twisting angle in a local coordinate system is illustrated consistent with the present inventive concepts. The schematic plot shown relates to finding a twisting angle of a functional assembly 150 of a catheter 100, including one or more coils 310 and/or electrodes 155, as described in reference to FIG. 1 and otherwise herein. System 10 can be configured to localize functional assembly 150 within a 3D coordinate system that is established by navigation module 250, as described herein. In a body cavity, the linearity of an impedance-based localization field may not be uniform throughout the body cavity, and thus, the estimation of the position of one or more electrodes 155 that is calculated by the z-loc algorithm, using a regionally linear assumption may be less accurate than a refined estimation that is calculated using other methods described herein, for example the h-loc algorithm described herein. In some embodiments, when catheter 100 (e.g., a mapping catheter), such as a catheter 100 comprising one or more fiducial pairs of coils 310 and electrodes 155, as described herein, is used to traverse the heart chamber for collecting the information of one or more impedance-based localization fields (e.g., such that electrodes 155 can be localized, as described herein), system 10 can calculate an average error for the z-loc estimation. If an error that is above a threshold is identified (e.g., an error in the z-loc estimation for one or more grid cells of a voxel grid), system 10 can be configured to update the impedance conversion matrix for the identified grid cells. FIG. 12 illustrates two examples of calculating an average error, such as for a catheter 100 comprising a basket-shaped functional assembly 150 with two or more 5-DOF coils 310 (or at least one 6-DOF coil 310), and for a catheter 100 comprising a linear functional assembly 150 with one 5-DOF or 6-DOF coil 310.

For a catheter 100 comprising a basket shaped functional assembly 150 with two 5-DOF coils 310, system 10 can calculate an optimal twisting angle based on model fitting and impedance measurements. With this optimal twisting angle θ_{twi}, the measured catheter direction θ_{dir}, and the measured translation \vec{X}ₜᵣₐₙₛₗₐₜᵢₒₙ from the magnetic origin, system 10 can map the z-loc estimation \vec{X}_{ei,est} back to a local coordinate system as follows:

$${\vec{X}}_{e i , est , loc} = {R}_{t w i}^{- 1} \cdot {R}_{d i r}^{- 1} \cdot ( {\vec{X}}_{e i , e s t} - {\vec{X}}_{t r a n s l a t i o n} ) \qquad ( 7 )$$

Here, R_{twi} is the transformation matrix based on θ_{twi}, R_{dir} is the transformation matrix based on θ_{dir}, and \vec{X}ₜᵣₐₙₛₗₐₜᵢₒₙ is the measured position of a first coil 310 positioned at the distal end of functional assembly 150. In the local coordinate system, the electrode position \vec{X}_{ei,loc} can be accurately calculated based on the length of functional assembly 150 (e.g., a basket shaped functional assembly 150). The distance between the z-loc estimation and the ground truth for each electrode is

$${d}_{i} = \vert {\vec{X}}_{e i , est , loc} - {\vec{X}}_{ei , loc} \vert ,$$

and the average error of z-loc estimation is

$${\vec{d}}_{z l o c} = \frac{{\sum }_{i = 1}^{N} {d}_{i}}{N} ,$$

where N is the number of electrodes. If d̅_{zloc} is larger than a preset threshold, system 10 can determine the scaling matrices used for the z-loc estimation are inaccurate, and should be updated.

Similarly, for a catheter 100 comprising a linear functional assembly 150 including at least one 5-DOF and/or 6-DOF coil 310, system 10 can calculate the true position \vec{X}ₑᵢ for an electrode 155, given the measured catheter direction, the position of the coil 310, and an identified catheter model, as described herein. With the z-loc estimation \vec{X}_{ei,est} for each electrode 155, system 10 can calculate the distance between the z-loc estimation and the ground truth for each electrode to be

$${d}_{i} = \vert {\vec{X}}_{e i , est , loc} - {\vec{X}}_{ei , loc} \vert .$$

System 10 can use the average error of the z-loc estimation

$${\tilde{d}}_{z l o c} = \frac{{\sum }_{i = 1}^{N} {d}_{i}}{N}$$

and a preset threshold to evaluate if the scaling matrices used for the z-loc estimation are accurate, or if the scaling matrices should be updated.

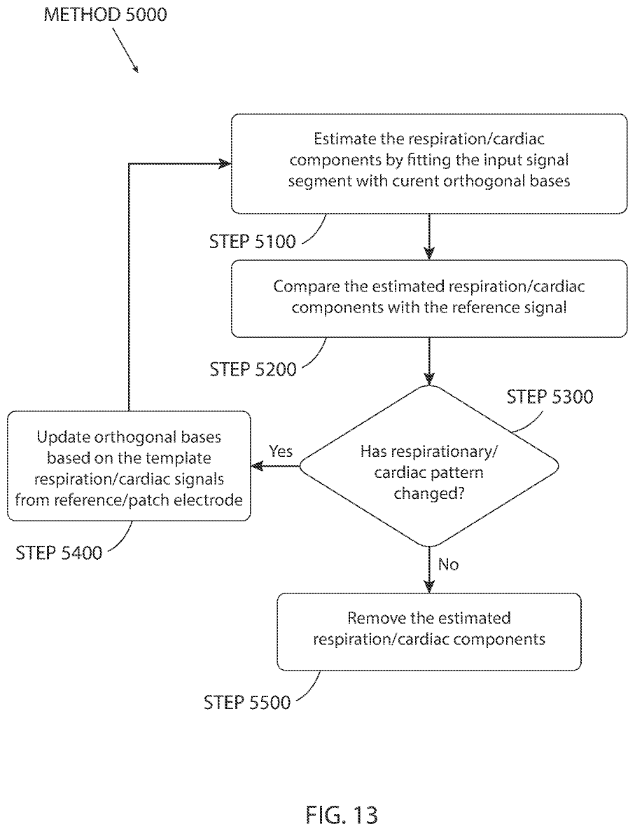

Referring now to FIG. 13, a flowchart of a method of compensating for respiration and cardiac motion is illustrated, consistent with the present inventive concepts. Method 5000 of FIG. 13 can be performed using the components of system 10 described in reference to FIG. 1 and otherwise herein. Respiratory motion and/or cardiac motion can both introduce undesired motion artifacts on both the magnetic localization and the impedance-based localization methods of system 10 described herein. Such motion can cause an artifactual motion in the visualization (e.g., when a localized device is displayed to a user) of the localized device (e.g., catheter 100) that is discordant with the true position of the device, for example relative to the heart surface. In addition, the measured impedance-based localization information of an electrode (e.g., an electrode 155) can vary when the physical position of the electrode is stationary relative to the cardiac surface. This variation also can cause an artifactual motion of the electrode. For example, such variation can occur during respiration, when the impedance of the human torso increases and decreases, as the patient inhales and exhales air, respectively. The overall impedance of the torso changes in proportion to the change in volume of the lungs.

When localizing a device, as described herein, it is essential to exclude the undesired respiration and cardiac motion artifacts to retain the true location of the device and maintain the accuracy navigation. In some embodiments, respiration and/or cardiac motion artifacts can affect both magnetic localization and impedance-based information in both time domain and the frequency domain.

In some embodiments, system 10 includes one or more respiration and/or cardiac motion compensation algorithms that are configured to resolve the respiration and/or cardiac motion artifacts in the magnetic localization information and/or impedance-based localization information. Compensation of the respiration and/or cardiac artifacts can be achieved by subtracting a signal reconstructed with orthogonal bases that represent the artifacts, for example such that only the true catheter movement is retained.

In some embodiments, cardiac motion artifacts are compensated using frequency selective filters. The filters can include spectral characteristics that remove the frequencies related to cardiac motion components in the frequency domain. In some embodiments, the frequency selective filter can be a FFT low pass filter that removes frequency components higher than 1 Hz.

Respiratory and/or cardiac motion can be periodic, or at least quasiperiodic, such that artifacts in the magnetic localization information and/or impedance-based localization information are also periodic. An optimized estimation of the respiration and cardiac motion can be found by fitting a periodic or quasiperiodic signal e(t) to the impedance and/or position measurement ƒ(t) of an electrode (e.g., electrode 155), such that

$${‖ f ( t ) - e ( t ) ‖}_{2}^{2}$$

is minimized. The periodic or quasiperiodic signal e(t) can be a summation of ƒ(t)'s projections on specially designed orthogonal bases that comprise periodic signals qₖ(t), k=1, 2, 3 . . . such that § qᵢ(t)qⱼ(t)dt=0, ∀i, j=1, 2, 3, . . . and i≠j. In some embodiments, the orthogonal bases can comprise sinusoidal functions from Fourier expansions, such that ƒ(t)'s projection on k-th basis

$${q}_{k} ( i ) = sin ( 2 π * k * f * t + {φ}_{k} ) can be {a}_{k} * sin ( 2 π * k * f * t + {φ}_{k} ) ,$$

$$k = 1 , 2 , 3 , …$$

Then, the estimated e(t)=Σₖ aₖ*qₖ(t) can be computed by minimizing

$${min}_{t} {‖ f ( t ) - {\sum }_{k} {a}_{k} * {q}_{k} ( t ) ‖}_{2}^{2} .$$

In some embodiments, the orthogonal bases can comprise signals that are mathematically orthogonal to a template of the respiration or cardiac motion signal.

In some embodiments, an adaptive method for respiration and cardiac compensation is performed, as illustrated by Method 5000 of FIG. 13. The optimal adaptive estimation of the respiration or cardiac components e (t) can be computed based on a segment of ƒ(t) within a moving window to match the respiration and cardiac motion varying along time. To keep the orthogonality of the bases for the segment of ƒ(t) within the moving window, the window length N should be selected such that

$${\int }_{t = {t}_{0}}^{{t}_{0} + N - 1} {q}_{i} ( t ) {q}_{j} ( t ) dt = 0 , ∀ i , j = 1 , 2 , 3 , …$$

and i≠j, which leads to a requirement for

$$N = lcm ( \frac{{T}_{0}}{i + j} , \frac{{T}_{0}}{i - j} )$$

for all i, j=1, 2, 3, . . . and i≠j.

In Step 5100 of Method 5000, system 10 can estimate the respiration and/or cardiac motion component of a localization signal by fitting the input signal with a current set of orthogonal bases. In Step 5200, the estimated respiration and/or cardiac motion components of the signal can be compared with a reference signal. In Step 5300, the algorithm determines if the respiratory pattern and/or pattern of cardiac motion has changed (e.g., changed since the orthogonal bases have been determined). In Step 5400, if it is determined that the pattern has changed, the orthogonal bases are updated based on template respiration and/or cardiac motion signals, such as signals recorded from coil 41, described herein. If the patterns have not changed, in Step 5500, the current orthogonal bases can be used to remove estimated respiration and/or cardiac motion components from localization signals, as described herein.

The above-described embodiments should be understood to serve only as illustrative examples; further embodiments are envisaged. Any feature described herein in relation to any one embodiment may be used alone, or in combination with other features described, and may also be used in combination with one or more features of any other of the embodiments, or any combination of any other of the embodiments. Furthermore, equivalents and modifications not described above may also be employed without departing from the scope of the inventive concepts, which are defined in the accompanying claims.

1. A system for performing a medical procedure on a patient, the system comprising:
a catheter device comprising a distal portion including a distal end and a functional assembly located on the distal portion of the catheter, the functional assembly comprising one or more electrodes; and
a console for operating the catheter device comprising:
a diagnostic module configured to record signals from at least one of the one or more electrodes; and
a processing unit configured to process the recorded signals.

2.-76. (canceled)
